(19) United States
(12) Patent Application Publication
(10) Pub. No.: US 2025/0114371 A1
(43) Pub. Date: Apr. 10, 2025

(54) BIOMARKER AND PATIENT SELECTION IN TREATMENT FOR MYELOFIBROSIS

(71) Applicant: GlaxoSmithKline LLC, Wilmington, DE (US)

(72) Inventors: Bryan William STROUSE, Wilmington, DE (US); Mei HUANG, Wilmington, DE (US)

(21) Appl. No.: 18/291,303

(22) PCT Filed: Jul. 29, 2022
(86) PCT No.: PCT/US2022/074301

§ 371 Date: Jan. 23, 2024

Related U.S. Application Data
(60) Provisional application No. 63/227,554, filed on Jul. 30, 2021.

(51) Int. Cl. A61K 31/5377; A61K 31/506; A61K 31/519; A61K 45/06; A61P 35/04; G01N 33/68

(52) U.S. Cl. CPC A61K 31/5377; A61K 31/506; A61K 31/519; A61K 45/06; A61P 35/04; G01N 33/68; G01N 2800/52

(57) ABSTRACT

Transfusion independence in, and overall survival of, myelofibrosis patients can be improved by selecting a patient specific treatment method. A patient's baseline ferritin level can be used as a biomarker to select a first line treatment. The patient's ferritin levels during treatment can inform decisions about terminating treatment or changing treatments to maintain transfusion dependence and/or convert a patient to transfusion independent from transfusion requiring or transfusion dependent.

### FIELD

This invention relates to methods of selecting first and/or second line treatment for myelofibrosis using the ferritin levels of the subject prior to and during treatment for myelofibrosis. In particular a patient's ferritin levels provide a biomarker that can be used to determine when a patient should be treated using momelotinib, a compound known to treat anemia and help patients gain or maintain transfusion independence.

### BACKGROUND

Myelofibrosis (MF) is a disease that affects approximately 40,000 to 50,000 patients worldwide, of which 70-80% of patients are categorized as intermediate to high risk MF patients. The median survival for all patients with MF is about 6 years, but is considerably worse for patients classified as intermediate-2 or high-risk MF, at 4 years and 2.25 years, respectively.

Myelofibrosis may occur de novo as Primary MF (PMF) or may arise from a pre-existing myeloproliferative neoplasm (MPN), primarily polycythemia vera (PV) or essential thrombocythemia (ET). Once these conditions reach the overtly fibrotic stage, they are virtually indistinguishable clinically.

The three cardinal disease manifestations of MF are (1) anemia, often in association with thrombocytopenia or other cytopenias; (2) constitutional symptoms, such as fatigue, night sweats, fever, cachexia, bone pain, pruritus, and weight loss; and (3) organomegaly due to extramedullary hematopoiesis, principally of the spleen and less often the liver, which can cause commonly associated symptoms such as abdominal distension and pain, early satiety, dyspnea, and diarrhea.

Ruxolitinib (RUX) is a Janus kinase (JAK) inhibitor used for the treatment of intermediate and high-risk myelofibrosis, including primary myelofibrosis, post-polycythemia vera myelofibrosis and post-essential thrombocythemia myelofibrosis. Ruxolitinib is used for treating approximately 70% of presenting patients, but is not approved for patients with severe thrombocytopenia.

Anemia and transfusion dependence are associated with reduced overall survival in patients with myelofibrosis. Approximately 60% of patients with myelofibrosis (MF) are anemic and 45% are transfusion dependent within 1 year of diagnosis, with most progressing to transfusion dependency overtime.[Pardanani el al. 2013 Am. J. Hematol. 88(4) pp. 312-316] Elevated hepcidin, moderate to severe anemia, and transfusion dependency are negative prognostic factors in MF, affecting overall survival (OS) and quality of life.[Nicolosi et al. 2018 Leukemia 32(5) pp. 1254-1258]

Approved JAK inhibitors (JAKi) provide spleen and symptom improvements but are generally myelosuppressive and do not address transfusion dependence (TD). Momelotinib (MMB) is a potent JAK1, JAK2 and ACVR1/ALK2 inhibitor with clinical activity against anemia, symptoms and splenomegaly in MF, as demonstrated in the previously conducted Phase 3 SIMPLIFY-1 & -2 trials. The SIMPLIFY 1 (S1) trial compared MMB and ruxolitinib (RUX) in JAKi-naïve patients (NCT01969838). [Mesa et al. 2017 J. of Clin. One. 35(34) pp. 3844-3850] The SIMPLIFY 2 (S2) trial compared MMB vs best available therapy (BAT; RUX in 88% of patients) in patients with prior RUX therapy (NCT02101268). [Harrison et al. 2018 Lancet. 5(2) pp. e73-e81]

MMB has demonstrated anemia benefit in direct comparison to RUX for JAKi naïve and previously RUX treated patients. Preclinical and clinical translational studies have demonstrated MMB's ability to address anemia and transfusion dependency is mechanistically linked to its differentiated suppression of ACVR1/ALK2-mediated hepcidin production [Asshoff et al. 2017 Blood. 129(13) pp. 1923-1830 and Oh et al. 2020 Blood Adv. 4(18) pp. 4282-4291]

Transfusion independence, and especially transfusion independence at week 24 of treatment, is associated with improved overall survival in myelofibrosis patients. A need exists for methods of determining or estimating a week 24 transfusion independent response in myelofibrosis patients and for methods of treating patients with myelofibrosis that will provide a week 24 transfusion independent response.

### SUMMARY

In accordance with the description, the following embodiments are provided.

Embodiment 1 is a method of treating myelofibrosis in a subject, the method comprising:
administering a therapeutically effective amount of momelotinib or a pharmaceutically acceptable salt thereof to a subject identified as having (i) myelofibrosis, and (ii) a ferritin level greater than 90 ng/mL.

Embodiment 2 is the method of embodiment 1, wherein the subject is identified as having a ferritin level greater than 90 ng/mL and less than 650 ng/mL.

Embodiment 3 is the method of embodiment 1, wherein the subject is identified as having a ferritin level greater than 650 ng/mL.

Embodiment 4 is the method of embodiment 3, further comprising administering a therapeutically effective amount of a second therapeutic agent to the subject.

Embodiment 5 is the method of any one of embodiments 1 to 4, further comprising determining the level of ferritin in a sample of a subject having myelofibrosis.

Embodiment 6 is the method of any one of embodiments 1 to 5, wherein the momelotinib or a pharmaceutically acceptable salt thereof is administered for a treatment period of a plurality of weeks.

Embodiment 7 is the method of embodiment 6, wherein the treatment period of a plurality of weeks is 12 weeks or more.

Embodiment 8 is the method of embodiment 6, wherein the treatment period of a plurality of weeks is 24 weeks or more.

Embodiment 9 is the method of embodiment 6, wherein the treatment period of a plurality of weeks is 36 weeks or more.

Embodiment 10 is the method of any one of embodiments 1 to 9, further comprising the steps of determining the level of ferritin in a sample of the subject once a month, or twice a month, or once weekly during a treatment period.

Embodiment 11 is the method of embodiment 10, wherein the level of ferritin in a sample of the subject is measured once weekly.

Embodiment 12 is the method of embodiment 10 or 11, wherein the subject is assessed as maintaining a ferritin level within a predetermined range during the treatment period.

Embodiment 13 is the method of embodiment 10 or 11, wherein the subject is assessed as having a ferritin level that is not within a predetermined range during the treatment period.

Embodiment 14 is the method of embodiment 10 or 11, wherein the subject is assessed as having a ferritin level that is not within a predetermined range during the treatment period and wherein the method further comprises the step of terminating administration of the momelotinib or a pharmaceutically acceptable salt thereof, and/or the optional second therapeutic agent.

Embodiment 15 is the method of embodiment 10 or 11, wherein the subject is assessed as having a ferritin level that is not within a predetermined range during the treatment period and wherein the method further comprises the steps of
terminating administration of the momelotinib or a pharmaceutically acceptable salt thereof, and/or the optional second therapeutic agent; and
administering a second line treatment comprising:
administering a therapeutically effective amount of a JAK inhibitor to the subject wherein the subject is assessed as having a ferritin level less than 90 ng/mL; or
administering a therapeutically effective amount of momelotinib or a pharmaceutically acceptable salt thereof to the subject wherein the subject is assessed as having a ferritin level greater than 90 ng/mL and less than or equal to 650 ng/mL; or
administering a therapeutically effective amount of momelotinib or a pharmaceutically acceptable salt thereof, and optionally a second therapeutic agent, to the subject wherein the subject is assessed as having a ferritin level greater than 650 ng/mL.

Embodiment 16 is the method of any one of embodiments 12 to 15, wherein the predetermined range of ferritin level during the treatment period is greater than or equal to 90 ng/mL, or wherein the predetermined range of ferritin level during the treatment period is greater than or equal to 90 ng/mL and less than or equal to 650 ng/mL.

Embodiment 17 is the method of any one of embodiments 12 to 15, wherein the predetermined range of ferritin level during the treatment period is greater than 650 ng/mL.

Embodiment 18 is a method of maintaining transfusion independence in a subject being treated for myelofibrosis, the method comprising
administering a therapeutically effective amount of a JAK inhibitor to a subject identified as (i) having myelofibrosis, (ii) being transfusion independent, and (iii) as having a ferritin level less than 90 ng/mL; or
administering a therapeutically effective amount of momelotinib or a pharmaceutically acceptable salt thereof to a subject identified as (i) having myelofibrosis, (ii) being transfusion independent, and (iii) as having a ferritin level greater than 90 ng/mL and less than or equal to 650 ng/mL; or
administering a therapeutically effective amount of momelotinib or a pharmaceutically acceptable salt thereof, and optionally a second therapeutic agent, to a subject identified as (i) having myelofibrosis, (ii) being transfusion independent, and (iii) as having a ferritin level greater than 650 ng/mL.

Embodiment 19 is a method of converting a subject being treated for myelofibrosis from transfusion requiring or from transfusion dependent to transfusion independent, the method comprising
administering a therapeutically effective amount of a JAK inhibitor to a subject identified as (i) having myelofibrosis, (ii) being transfusion requiring or transfusion dependent, and (iii) as having a ferritin level less than 90 ng/mL; or
administering a therapeutically effective amount of momelotinib or a pharmaceutically acceptable salt thereof to a subject identified as (i) having myelofibrosis, (ii) being transfusion requiring or transfusion dependent, and (iii) as having a ferritin level greater than 90 ng/mL;
administering a therapeutically effective amount of momelotinib or a pharmaceutically acceptable salt thereof to a subject identified as (i) having myelofibrosis, (ii) being transfusion requiring or transfusion dependent, and (iii) as having a ferritin level greater than 90 ng/mL and less than or equal to 650 ng/mL; or
administering a therapeutically effective amount of momelotinib or a pharmaceutically acceptable salt thereof, and optionally a second therapeutic agent, to a subject identified as (i) having myelofibrosis, (ii) being transfusion requiring or transfusion dependent, and (iii) as having a ferritin level greater than 650 ng/mL.

Embodiment 20 is the method of embodiment 18 or 19, wherein the JAK inhibitor is chosen from momelotinib or a pharmaceutically acceptable salt thereof, ruxolitinib or fedratinib.

Embodiment 21 is the method of embodiment 20, wherein the JAK inhibitor is momelotinib or a pharmaceutically acceptable salt thereof or ruxolitinib.

Embodiment 22 is the method of embodiment 20, wherein the JAK inhibitor is ruxolitinib.

Embodiment 23 is the method of embodiment 20, wherein the JAK inhibitor is momelotinib or a pharmaceutically acceptable salt thereof.

Embodiment 24 is the method of any one of embodiments 4, or 18 to 23, wherein the second therapeutic agent is a BET protein inhibitor, or is a BRD4 inhibitor.

Embodiment 25 is the method of embodiment 24, wherein the second therapeutic agent is chosen from GSK2820151. GSK525762, GS-5829, R06870810 (IV), BAY1238097, CC-90010, BMS-986158, 1NCB054329, 1NCB057643, ODM-207, AZD5153, FT-1101, ABBV-744, ABBV-075. PLX51107. B1894999, OTX015/MK8628, ZEN003694, RVX-000222, CPI-0610, apabetalone and fedratinib.

Embodiment 26 is a method of treating or preventing anemia in a subject being treated for myelofibrosis comprising
administering a therapeutically effective amount of momelotinib or a pharmaceutically acceptable salt thereof to a subject identified (i) as having myelofibrosis, and (ii) as having anemia or as being at risk for becoming anemic, and (iii) as having a ferritin level greater than or equal to 90 ng/mL.

Embodiment 27 is the method of any one of embodiments 18 to 26, further comprising the earlier step of determining the level of ferritin in a sample of a subject having myelofibrosis.

Embodiment 28 is the method of any one of embodiments 18 to 27, wherein the momelotinib or a pharmaceutically acceptable salt thereof is administered for a treatment period of a plurality of weeks.

Embodiment 29 is the method of embodiment 28, wherein the treatment period of a plurality of weeks is 12 weeks or more.

Embodiment 30 is the method of embodiment 28, wherein the treatment period of a plurality of weeks is 24 weeks or more.

Embodiment 31 is the method of embodiment 28, wherein the treatment period of a plurality of weeks is 36 weeks or more.

Embodiment 32 is the method of any one of embodiments 18 to 31, further comprising the steps of determining the level of ferritin in a sample of the subject once a month, or twice a month, or once weekly during a treatment period.

Embodiment 33 is the method of embodiment 32, wherein the level of ferritin in a sample of the subject is measured weekly.

Embodiment 34 is the method of embodiment 32 or 33, wherein the subject is assessed as maintaining a ferritin level within a predetermined range during the treatment period.

Embodiment 35 is the method of embodiment 32 or 33, wherein the subject is assessed as having a ferritin level that is not within a predetermined range during the treatment period.

Embodiment 36 is the method of embodiment 32 or 33, wherein the subject is assessed as having a ferritin level that is not within a predetermined range during the treatment period and wherein the method further comprises the step of terminating administration of the JAK inhibitor, or the momelotinib or a pharmaceutically acceptable salt thereof, and/or the optional second therapeutic agent.

Embodiment 37 is the method of embodiment 32 or 33, wherein the subject is assessed as having a ferritin level that is not within a predetermined range during the treatment period and wherein the method further comprises the steps of
terminating administration of the JAK inhibitor, or the momelotinib or a pharmaceutically acceptable salt thereof, and/or the optional second therapeutic agent; and
administering a second line treatment comprising:
   administering a therapeutically effective amount of a JAK inhibitor to the subject wherein the subject is assessed as having a ferritin level less than 90 ng/mL; or
   administering a therapeutically effective amount of momelotinib or a pharmaceutically acceptable salt thereof to the subject wherein the subject is assessed as having a ferritin level greater than 90 ng/mL; or
   administering a therapeutically effective amount of momelotinib or a pharmaceutically acceptable salt thereof to the subject wherein the subject is assessed as having a ferritin level greater than 90 ng/mL and less than or equal to 650 ng/mL; or
   administering a therapeutically effective amount of momelotinib or a pharmaceutically acceptable salt thereof, and optionally a second therapeutic agent, to the subject wherein the subject is assessed as having a ferritin level greater than 650 ng/mL.

Embodiment 38 is the method of any one of embodiments 34 to 37, wherein the predetermined range of ferritin level during the treatment period is greater than or equal to 90 ng/mL, or wherein the predetermined range of ferritin level during the treatment period is greater than or equal to 90 ng/mL and less than or equal to 650 ng/mL.

Embodiment 39 is the method of any one of embodiments 34 to 37, wherein the predetermined range of ferritin level during the treatment period is greater than 650 ng/mL.

Embodiment 40 is the method of any one of embodiments 1 to 39, wherein the subject is a human.

Embodiment 41 is the method of any one of embodiments 1 to 40, wherein the subject is an adult human.

Embodiment 42 is the method of any one of embodiments 1 to 41, wherein the subject has previously been treated with JAK inhibitor therapy other than momelotinib.

Embodiment 43 is the method of embodiment 42, wherein the subject has previously been treated with ruxolitinib.

Embodiment 44 is the method of embodiment 42 or 43, wherein the subject has had an inadequate response to or is intolerant of ruxolitinib.

Embodiment 45 is the method of any one of embodiments 42 to 44, wherein the subject failed to respond or ceased to respond to previous ruxolitinib therapy.

Embodiment 46 is the method of any one of embodiments 1 to 41, wherein the subject is naïve to JAK inhibitor therapy.

Embodiment 47 is the method of any one of embodiments 1 to 46, wherein the momelotinib or pharmaceutically acceptable salt thereof is momelotinib dihydrochloride salt.

Embodiment 48 is the method of any one of embodiments 1 to 46, wherein the momelotinib or pharmaceutically acceptable salt thereof is momelotinib dihydrochloride monohydrate.

Embodiment 49 is the method of any one of embodiments 1 to 46, wherein the momelotinib or pharmaceutically acceptable salt thereof is momelotinib dihydrochloride monohydrate Form II.

Embodiment 50 is the method of any one of embodiments 1 to 49, wherein the momelotinib or pharmaceutically acceptable salt thereof is provided in a pharmaceutically acceptable composition.

Embodiment 51 is the method of any one of embodiments 1 to 50, wherein the therapeutically effective amount is between 50 mg/day and 200 mg/day.

Embodiment 52 is the method of embodiment 51, wherein the therapeutically effective amount is 200 mg/day, or 150 mg/day, or 100 mg/day, or 50 mg/day.

Embodiment 53 is the method of any one of embodiments 1 to 52, wherein the momelotinib or pharmaceutically acceptable salt thereof is administered orally.

Embodiment 54 is the method of any one of embodiments 1 to 53, wherein the momelotinib or pharmaceutically acceptable salt thereof is administered daily.

Embodiment 55 is the method of embodiment 54, wherein the momelotinib or pharmaceutically acceptable salt thereof is administered once daily.

A further aspect of the present disclosure, is momelotinib for use in the methods of treatment disclosed herein. Another aspect of the present disclosure is the use of momelotinib in the manufacture of a medicament for use in the methods of treatment disclosed herein. Additional objects and advantages will be set forth in part in the description which follows, and in part will be understood from the description, or may be learned by practice. The objects and advantages will be realized and attained by means of the elements and combinations particularly pointed out in the appended claims. It is to be understood that both the foregoing general description and the following detailed description are exemplary and explanatory only and are not restrictive of the claims.

The accompanying drawings, which are incorporated in and constitute a part of this specification, illustrate one (several) embodiment(s) and together with the description, serve to explain the principles described herein.

### BRIEF DESCRIPTION OF THE DRAWINGS

FIG. 1 shows results are from the SIMPLIFY-1 trial. The rate of week 24 transfusion independence in subjects treated with momelotinib (MMB) compared to subjects treated with ruxolitinib (RUX) with subjects grouped by platelet counts (PLT) less than 150×10⁹/L, less than 300×10⁹/L, and greater than 300×10⁹/L.

FIG. 2 shows results are from the SIMPLIFY-1 trial. The rate of week 24 transfusion independence in subjects treated with momelotinib compared to subjects treated with ruxolitinib is shown for subjects grouped with hemoglobin levels (Hgb) less than 8 g/dL, less than 10 g/dL, less than 12 g/dL, less than 14 g/dL, and greater than 14 g/dL.

FIG. 3 shows results are from the SIMPLIFY-1 trial. The rate of week 24 transfusion independence in subjects treated with momelotinib compared to subjects treated with ruxolitinib is shown for subjects grouped by transfusion independent (TI), transfusion requiring (TR) and transfusion dependent (TD).

FIG. 4 compares the percentage of myelofibrosis subjects surviving after treatment with MMB alone or after a first treatment with RUX and a cross over to treatment with MMB in the SIMPLIFY-1 trial.

FIG. 5 compares the percentage of myelofibrosis subjects surviving after treatment with MMB alone or after a first treatment with the best available therapy (BAT). RUX, and a cross over to treatment with MMB in the SIMPLIFY-2 trial.

FIG. 6 shows the overall survival in all patients randomized to MMB by Week 24 TI Response in the SIMPLIFY-1 trial. The graph shows the survival of transfusion independent responder myelofibrosis subjects to the survival of transfusion independent nonresponder myelofibrosis subjects over time after MMB-randomization.

FIG. 7 shows the overall survival in anemic patients randomized to MMB by Week 24 TI Response in the SIMPLIFY-1 trial. The graph compares the survival of transfusion independent responder myelofibrosis subjects to the survival of transfusion independent nonresponder myelofibrosis subjects over time after MMB-randomization.

FIG. 8 shows the overall survival in all patients randomized to MMB by Week 24 TI Response in the SIMPLIFY-2 trial. The graph compares the survival of transfusion independent responder myelofibrosis subjects to the survival of transfusion independent nonresponder myelofibrosis subjects over time after MMB-randomization.

FIG. 9 shows the overall survival in spleen responder patients randomized to MMB by Week 24 spleen response in the SIMPLIFY-1 trial. The graph compares the survival of all spleen responder myelofibrosis subjects to the survival of spleen nonresponder myelofibrosis subjects over time.

FIG. 10 shows the overall survival in symptom responder (TSS) patients randomized to MMB by Week 24 symptom response in the SIMPLIFY-1 trial. The graph compares the survival of all symptom responder myelofibrosis subjects to the survival of symptom nonresponder myelofibrosis subjects over time.

FIG. 11 shows the mean ferritin levels in transfusion independent responder subjects compared to transfusion independent nonresponder subjects over 24 weeks of treatment in the “1672 Study.”

FIG. 12 shows baseline ferritin and change from baseline at week 12 by transfusion independent response at week 24 in MMB subjects from SIMPLIFY-1.

FIG. 13 shows baseline ferritin and change from baseline at week 12 by transfusion independent response at week 24 in RUX subjects from SIMPLIFY-1.

FIG. 14 shows transfusion independent response at week 24 by baseline ferritin level in SIMPLIFY-1 trial subjects.

FIG. 15 shows transfusion independent response at week 24 by baseline ferritin level in SIMPLIFY-2 trial subjects.

### DESCRIPTION

### I. Definitions

It is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

It must be noted that, as used in the specification and the appended claims, the singular forms “a,” “an” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

As used herein the term “anemia response” refers to an increase in the patients hemoglobin level or a patient who was transfusion dependent becoming transfusion independent. Desirably, a minimum increase in hemoglobin of 0.5 g/dL, such as 1.0 g/dL 1.5 g/dL or 2.0 g/dL lasting a minimum of 8 weeks is achieved, which is the level of improvement specified in the International Working Group (IWG) consensus criteria. However, smaller, but still medically significant, increases in hemoglobin are also considered to be within the term “anemia response.”

As used herein the term “transfusion dependent” (TD) refers to a subject's need for 4 or more units of red blood cells (RBCs) or has a hemoglobin (Hgb) level, of less than or equal to 8 g/dL in the 8 weeks prior to the start of treatment.

As used herein the term “transfusion independent” or “Transfusion Independence” (TI) refers to a subject who required no red blood cell (RBC) transfusions over a 12 week period and had no hemoglobin levels less than 8 g/dL to 8.5 g/dL over the 12 week period. In some embodiments, the 12 week period of transfusion independence is the terminal 12 weeks of a 24 week study period. By red blood cell transfusion independence rate at week 24 of a study is meant the proportion of subjects who were transfusion independent at week 24, excluding cases associated with clinically overt bleeding.

As used herein the term “transfusion requiring” (TR) refers to subject's whose transfusion status does not meet the requirements of either TD or TI.

As used herein the term “Week 24” or “WK24” refers to the 24^{th} week of treatment.

As used herein the term “Week 24 Transfusion Independence Response” (WK24 TI-R) refers to the response of a subject whose transfusion status is TI at the 24^{th} week after the start of treatment.

As used herein the term “W24 TI nonresponsive” or “W24 TI nonresponder” (TI-NR)” refers to subjects who do not achieve a W24 transfusion independent response (TI-R), which is defined by no RBC transfusion within ≥12 weeks prior to Week 24, with Hgb≥8 g/dL.

As used herein the term “W24 spleen response” refers to a subject's spleen having shown a 35% or more spleen volume reduction versus the volume at baseline (BL) or start of treatment.

As used herein the term “W24 symptom response” refers to 50% or greater reduction in a subject's MFSAF total symptom score versus BL or start of treatment.

As used herein the term “spleen response” refers to a reduction in the size of the patient's spleen as assessed by either palpation of a previously palpable spleen during physical exam or by diagnostic imaging. The IWG consensus criteria specifies that there be either a minimum 50% reduction in palpable splenomegaly (spleen enlargement) of a spleen that is at least 10 cm at baseline (prior to treatment) or of a spleen that is palpable at more than 5 cm below the left costal margin at baseline becomes not palpable. However, smaller reductions are also considered to be within the term “spleen response”. Splenic enlargement can be assessed by palpation. Splenic size and volume can also be measured by diagnostic imaging such as ultrasound, CT or MRI). In some cases, normal spleen size is considered to be approximately 11.0 cm, in craniocaudal length.

As used herein the term “symptom response” or “symptomatic response” refers to a reduction in a patient's average daily TSS of at least 50% as compared to a baseline TSS determined at or before initiation of treatment (e.g., as described herein).

As used herein the term “Treatment” or “treating” refers to an approach for obtaining beneficial or desired results including clinical results. Beneficial or desired clinical results may include one or more of the following: a) inhibiting the disease or condition (e.g., decreasing one or more symptoms or manifestations resulting from the disease or condition, and/or diminishing the extent of the disease or condition); b) slowing or arresting the development of one or more clinical symptoms associated with the disease or condition (e.g., stabilizing the disease or condition, preventing or delaying the worsening or progression of the disease or condition, and/or preventing or delaying the spread (e.g., metastasis) of the disease or condition); c) relieving the disease, that is, causing the regression of clinical symptoms (e.g., ameliorating the disease state, alleviating or ameliorating one or more symptoms or manifestations, providing partial or total remission of the disease or condition, enhancing effect of another medication, delaying the progression of the disease, increasing the quality of life, and/or prolonging survival; and/or d) improving or stabilizing or preventing decline in one or more clinical endpoints (e.g., as described here), including but not limited to, transfusion independence, conversion from transfusion dependent to independent, total symptom score (TSS), splenic response (SRR), and improvement of anemia.

The term “effective amount” refers to an amount that may be effective to elicit the desired biological or medical response, including the amount of a compound that, when administered to a subject for treating a disease, is sufficient to effect such treatment for the disease. The effective amount will vary depending on the compound, the disease and its severity and the age, weight, etc., of the subject to be treated. The effective amount can include a range of amounts.

The terms “subject” and “patient” are used interchangeably and refer to an animal, such as a mammal (including a human), that has been or will be the object of treatment, observation or experiment. The methods described herein may be useful in human therapy and/or veterinary applications. In some embodiments, the subject is a mammal. In one embodiment, the subject is a human. A variety of other mammals can be treated using the methods of the present disclosure. For instance, mammals including, but not limited to, cows, sheep, goats, horses, dogs, cats, guinea pigs, rats or other bovine, ovine, equine, canine, feline, rodent or murine species can be treated.

“Human in need thereof” refers to a human who may have or is suspected to have diseases or conditions that would benefit from certain treatment; for example, being treated with the compounds according to the present application. The terms “subject in need thereof” or “patient in need thereof” refer to a subject or a patient who may have, is diagnosed, or is suspected to have a disease, or disorder, or condition that would benefit from the treatment described herein.

The term “therapeutically effective amount” of a compound or a pharmaceutically acceptable salt, isomer, prodrug, or solvate thereof, means an amount sufficient to effect treatment when administered to a subject, e.g., to provide a therapeutic benefit such as amelioration of one or more symptoms or slowing of disease progression. The therapeutically effective amount may vary depending on the subject, and disease or condition being treated, the weight and age of the subject, the severity of the disease or condition, and the manner of administering, which can be readily determined.

### II. SIMPLIFY-1 and SIMPLIFY-2 Clinical Trials

Momelotinib (MMB) is a potent, selective, orally-bioavailable, small-molecule inhibitor of JAK1, JAK2 and ACVR1 that was developed for the treatment of myelofibrosis (MF).

In two Phase three clinical trials (SIMPLIFY-1 and -2), however, MMB failed to meet the pre-defined secondary endpoints of TSS response in SIMPLIFY-1 and the primary endpoint of SRR in SIMPLIFY-2.

In the SIMPLIFY-1 trial (NCT01969838; GS-US-352-0101), the efficacy and safety of MMB versus ruxolitinib (RUX) was studied in patients with myelofibrosis who were naïve to treatment with a JAK inhibitor. Patients (N=432) with high risk or intermediate-2 risk or symptomatic intermediate-1 risk myelofibrosis received 24 weeks of treatment with 200 mg MMB once daily or 20 mg RUX twice a day (or per label), after which all patients could receive open-label momelotinib. Efficacy was measured, with a goal of demonstrating non-inferiority of MMB to RUX, by spleen response, total symptom score (TSS), rate of red blood cell transfusion, and transfusion-independence or transfusion dependence. The primary endpoint was a reduction by at least 35% in the spleen volume at 24 weeks compared with baseline.

An initial analysis of the results of the SIMPLIFY 1 trial was reported by Mesa et al. (SIMPLIFY-1: A Phase III Randomized Trial of Momelotinib Versus Ruxolitinib in Janus Kinase Inhibitor-Naive Patients with Myelofibrosis”, J. Clinical Oncology 2017, 35(34):3844-3850). That analysis of the SIMPLIFY-1 trial data indicated momelotinib was noninferior to RUX for the reduction of spleen size in JAKi-naïve patients, thus meeting the study's primary endpoint. However, non-inferiority was not demonstrated for the secondary endpoint of total symptom score (TSS) response, despite evidence of momelotinib's symptomatic benefits in symptomatic patients in that study. MMB treatment was associated with an increased transfusion independence rate, a decreased transfusion dependence rate and a reduced transfusion rate compared to RUX, all of which were nominally statistically-significant.

In the SIMPLIFY-2 trial (NCT02101268; GS-US-352-1214), the efficacy and safety of MMB versus best available treatment (BAT) was studied in anemic or thrombocytopenic subjects with myelofibrosis who were previously treated with ruxolitinib (RUX). There was no lower limit for the required baseline platelet count. Efficacy, with a goal of demonstrating superiority of MMB over BAT, was measured by spleen response, total symptom score (TSS), rate of red blood cell transfusion, and transfusion-independence or transfusion dependence. The primary endpoint was a reduction by at least 35% in the spleen volume at 24 weeks compared with baseline.

An initial analysis of the results of the SIMPLIFY-2 trial was reported by Harrison et al. (“Momelotinib versus best available therapy in patients with myelofibrosis previously treated with ruxolitinib (SIMPLIFY 2): a randomized, open-label, phase 3 trial. Lancet Haematol; Volume 5, Issue 2, February 2018, Pages e73-e81). That analysis of the SIMPLIFY-2 trial data indicated momelotinib was not superior to BAT for the reduction of spleen size, thus the trial failed to achieve its primary endpoint.

Although the key secondary endpoints were nominally significant in the analysis by Harrison et al., theses were not considered statistically significant in the hierarchy of analysis endpoints. In general, patients in the momelotinib group had a greater total symptom score (TSS) response, fewer transfusions, higher transfusion independence, and lower transfusion dependence compared to patients in the BAT group. The initial analysis was complicated by the failure to mandate discontinuation of ruxolitinib prior to the start of randomized study treatment. By not including a mandatory washout from prior ruxolitinib, the evaluation of the splenic response was obscured in this study in subjects in either arm. In addition, patients enrolled in this study were not selected based on splenic progression on RUX.

A re-analysis of the data from the SIMPLIFY-1 and SIMPLIFY-2 trials discovered that momelotinib is effective in reducing spleen size (SSR), improving total symptom scores (TSS), and improving transfusion independence rates in patients whose platelet counts are 150×109 per liter or below, without momelotinib administration causing thrombocytopenia, and without therefore requiring dose reduction or interruption for thrombocytopenia.

Further analysis of data collected in these trials shows that patient baseline and on-treatment serum ferritin levels provide information about the patients likelihood of remaining transfusion independent, or becoming transfusion independent, when that patient is treated with momelotinib. Overall survival is improved in patients that remain transfusion independent.

### III. Transfusion Independence as a Predictive Tool

Myelofibrosis patients experience a number of disease related symptoms including decreased hemoglobin levels and elevated hepcidin levels, splenomegaly, cytopenia, and anemia. In almost all cases the patient will receive a blood transfusion, with some patients requiring repeated transfusions. Anemia and transfusion dependence are factors associated with reduced overall survival in patients with myelofibrosis.

Patients that remain transfusion independent show improved overall survival. It has been shown that patients that remain transfusion independent on the 24th week of treatment show the greatest overall survival benefits from treatment.

Determining Week 24 Transfusion Independence (WK24 TI) can be complex and simple markers for predicting or assessing WK24 TI were explored. Myelofibrosis patient hepcidin and hemoglobin levels were analyzed. The patient's hepcidin and/or hemoglobin level were not predictive of a patient's transfusion dependence or independence. However, ferritin levels in patients with myelofibrosis were found to be predictive of a patient's transfusion independence including their WK24 TI.

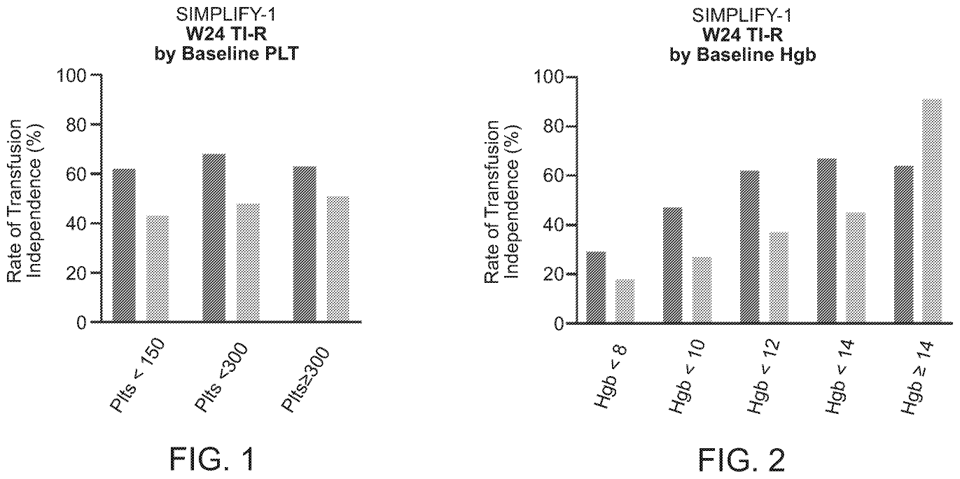

FIGS. 1, 2 and 3 show SIMPLIFY-1 (S1) Week 24 TI response rates for MMB compared to RUX by baseline characteristics. The W24 TI-R rate in S1 was higher in patients randomized to MMB vs RUX, irrespective of the degree of baseline anemia, or the baseline platelet (PLT) count or transfusion status. [EHA 2021 poster EP1081].

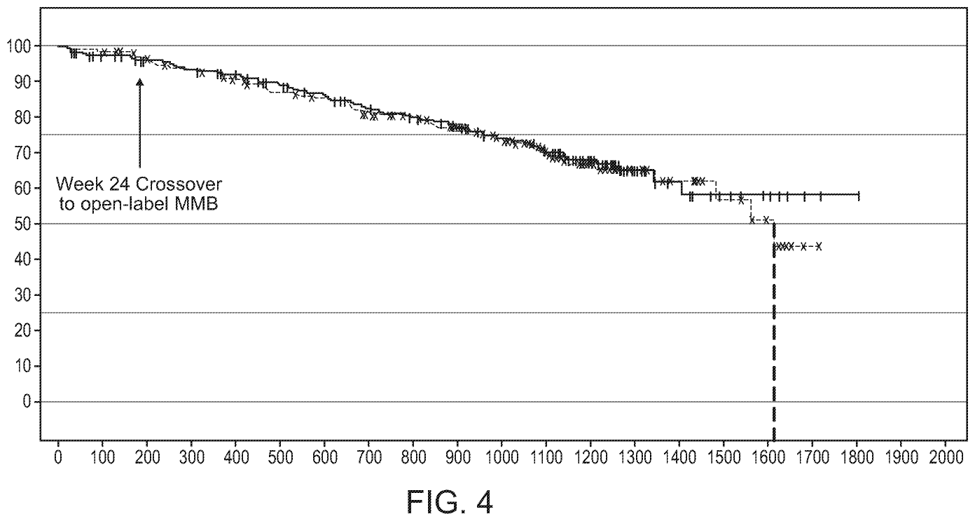

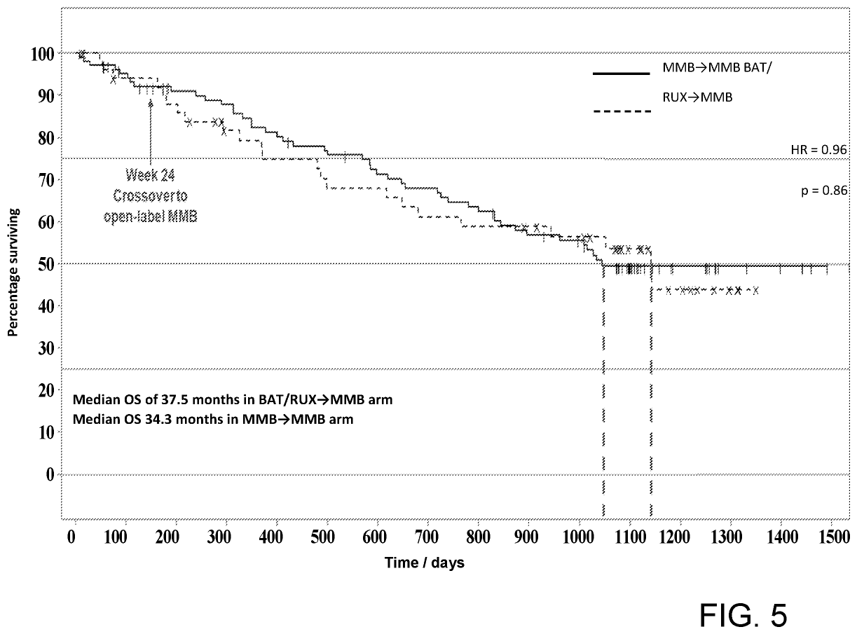

FIGS. 4 and 5 show survival in both JAKi-naïve and JAKi-exposed patients. S1 showed that in JAKi-naïve patients, robust survival was observed on extended treatment with MMB, regardless of starting therapy. S2 showed the best reported overall survival results for patients who have been previously treated with RUX. [Verstovsck et al. ASH. Presentation 2020]

### IV. Ferritin as a Biomarker for Patient Selection

Anemia in myelofibrosis patients is often treated with blood transfusions and repeated blood transfusions may cause iron overload in the liver and blood. Ferritin is a blood protein that stores the excess iron and may become elevated in patients that receive repeated transfusions. In some circumstances a patient's iron and ferritin levels may be correlated; however, the correlation of these two components can be lost in patients that experience inflammation, transfusions, or receive certain drugs. RUX is known to generally increase ferritin in patients that receive RUX.

Ferritin is measured using a routine blood test. Normal ferritin ranges for healthy humans range from 10 to 300 ng/mL. For purposes of this disclosure, blood ferritin levels of less than 90 ng/mL are considered a low to normal range. Blood ferritin levels of more than 90 ng/mL but less than or equal to 650 ng/mL are considered mid range to elevated. Blood ferritin levels of more than 650 ng/mL are elevated well outside the normal range.

Methods of selecting treatment for patients with myelofibrosis based on a readily measurable biomarker, especially biomarkers that correlate to or that estimate or predict a patient's likelihood of being transfusion independent after 24 weeks of treatment are disclosed herein. In some embodiments, a subject's baseline ferritin levels may be used to select a first line treatment. In some embodiments, a subject's on-treatment ferritin levels may be used to assess the continued viability of the first line treatment. In some embodiments, a subject's on-treatment ferritin levels may be used to select a second line treatment.

In some embodiments, the methods described herein comprise the step of determining the level of ferritin in a sample of a subject having myelofibrosis. In some embodiments the ferritin level is measure before treatment, i.e. a baseline level. In some embodiments, the methods described herein further comprise the steps of determining the level of ferritin in a sample of the subject once a month, or twice a month, or once weekly during a treatment period i.e., on-treatment ferritin levels. In some embodiments, the level of ferritin in a sample of the subject is measured weekly.

In some embodiments, the subject is assessed as maintaining a ferritin level within a predetermined range during the treatment period. In some embodiments, the subject is assessed as maintaining a ferritin level within a predetermined range during the treatment period and the treatment is continued.

In some embodiments, the subject is assessed as having a ferritin level that is not within a predetermined range during the treatment period. In some embodiments, when ferritin levels are outside of the range used to determine the first line treatment, that first line treatment may be terminated. In some embodiments, a second line treatment may then be selected. In some embodiments the second line treatment is different from the first line treatment.

In some embodiments, the subject's ferritin level is less than 90 ng/mL. In this range, in some embodiments, a treatment method comprises administration of a JAK inhibitor.

In some embodiments, the subject's ferritin level before or during the treatment period is greater than or equal to 90 ng/mL. In some embodiments, the subject's ferritin level before or during the treatment period is greater than or equal to 90 ng/mL and less than or equal to 650 ng/mL. When the subject's ferritin level is greater than or equal to 90 ng/mL, in some embodiments, a treatment method comprises administration of momelotinib.

In some embodiments, the subject's ferritin level before or during the treatment period is greater than or equal to 650 ng/mL. In this range, in some embodiments, a treatment method comprises administration of momelotinib. In this range, in some embodiments, a treatment method comprises administration of momelotinib combined with a second therapeutic agent.

### V. Methods of Treating Myelofibrosis

Provided herein are methods of treating myelofibrosis in a subject, the method comprising:
administering a therapeutically effective amount of momelotinib or a pharmaceutically acceptable salt thereof to a subject identified as having (i) myelofibrosis, and (ii) a ferritin level greater than 90 ng/mL.

In some embodiments, the subject is identified as having a ferritin level greater than 90 ng/mL and less than 650 ng/mL. In some embodiments, the subject is identified as having a ferritin level greater than 650 ng/mL. In some embodiments, the method further comprises administering a therapeutically effective amount of a second therapeutic agent to the subject.

Provided herein are methods of maintaining transfusion independence in a subject being treated for myelofibrosis, the method comprising
administering a therapeutically effective amount of a JAK inhibitor to a subject identified as (i) having myelofibrosis, (ii) being transfusion independent, and (iii) as having a ferritin level less than 90 ng/mL; or
administering a therapeutically effective amount of momelotinib or a pharmaceutically acceptable salt thereof to a subject identified as (i) having myelofibrosis, (ii) being transfusion independent, and (iii) as having a ferritin level greater than 90 ng/; or
administering a therapeutically effective amount of momelotinib or a pharmaceutically acceptable salt thereof to a subject identified as (i) having myelofibrosis, (ii) being transfusion independent, and (iii) as having a ferritin level greater than 90 ng/mL and less than or equal to 650 ng/mL; or
administering a therapeutically effective amount of momelotinib or a pharmaceutically acceptable salt thereof, and optionally a second therapeutic agent, to a subject identified as (i) having myelofibrosis, (ii) being transfusion independent, and (iii) as having a ferritin level greater than 650 ng/mL.

Provided herein are methods of converting a subject being treated for myelofibrosis from transfusion requiring or from transfusion dependent to transfusion independent, the method comprising
administering a therapeutically effective amount of a JAK inhibitor to a subject identified as (i) having myelofibrosis, (ii) being transfusion requiring or transfusion dependent, and (iii) as having a ferritin level less than 90 ng/mL; or
administering a therapeutically effective amount of momelotinib or a pharmaceutically acceptable salt thereof to a subject identified as (i) having myelofibrosis, (ii) being transfusion requiring or transfusion dependent, and (iii) as having a ferritin level greater than 90 ng/mL; or
administering a therapeutically effective amount of momelotinib or a pharmaceutically acceptable salt thereof to a subject identified as (i) having myelofibrosis, (ii) being transfusion requiring or transfusion dependent, and (iii) as having a ferritin level greater than 90 ng/mL and less than or equal to 650 ng/mL; or
administering a therapeutically effective amount of momelotinib or a pharmaceutically acceptable salt thereof, and optionally a second therapeutic agent, to a subject identified as (i) having myelofibrosis, (ii) being transfusion requiring or transfusion dependent, and (iii) as having a ferritin level greater than 650 ng/mL.

Provided herein are methods of treating or preventing anemia in a subject being treated for myelofibrosis comprising administering a therapeutically effective amount of momelotinib or a pharmaceutically acceptable salt thereof to a subject identified (i) as having myelofibrosis, and (ii) as having anemia or as being at risk for becoming anemic, and (iii) as having a ferritin level greater than or equal to 90 ng/mL.

Provided herein are methods of treating or preventing anemia in a subject being treated for myelofibrosis comprising administering a therapeutically effective amount of momelotinib or a pharmaceutically acceptable salt thereof to a subject identified (i) as having myelofibrosis, and (ii) as having anemia or as being at risk for becoming anemic, and (iii) as having a ferritin level greater than or equal to 90 ng/mL and less than or equal to 650 ng/mL.

In some embodiments, the method described herein further comprise the earlier step of determining the level of ferritin in a sample of a subject having myelofibrosis.

In some embodiments, the methods described herein further comprise the steps of determining the level of ferritin in a sample of the subject once a month, or twice a month, or once weekly during a treatment period. In some embodiments, the level of ferritin in a sample of the subject is measured weekly.

In some embodiments, the subject is assessed as maintaining a ferritin level within a predetermined range during the treatment period.

In some embodiments, the subject is assessed as having a ferritin level that is not within a predetermined range during the treatment period.

In some embodiments, the subject is assessed as having a ferritin level that is not within a predetermined range during the treatment period and the method further comprises the step of terminating administration of the JAK inhibitor, or the momelotinib or a pharmaceutically acceptable salt thereof, and/or the optional second therapeutic agent.

In some embodiments, the subject is assessed as having a ferritin level that is not within a predetermined range during the treatment period and the method further comprises the steps of
terminating administration of the JAK inhibitor, or the momelotinib or a pharmaceutically acceptable salt thereof, and/or the optional second therapeutic agent; and
administering a second line treatment comprising:
   administering a therapeutically effective amount of a JAK inhibitor to the subject wherein the subject is assessed as having a ferritin level less than 90 ng/mL; or
   administering a therapeutically effective amount of momelotinib or a pharmaceutically acceptable salt thereof to the subject wherein the subject is assessed as having a ferritin level greater than 90 ng/mL; or
   administering a therapeutically effective amount of momelotinib or a pharmaceutically acceptable salt thereof to the subject wherein the subject is assessed as having a ferritin level greater than 90 ng/mL and less than or equal to 650 ng/mL; or
   administering a therapeutically effective amount of momelotinib or a pharmaceutically acceptable salt thereof, and optionally a second therapeutic agent, to the subject wherein the subject is assessed as having a ferritin level greater than 650 ng/mL.

In some embodiments, the predetermined range of ferritin level during the treatment period is less than 90 ng/mL.

In some embodiments, the predetermined range of ferritin level during the treatment period is greater than or equal to 90 ng/mL.

In some embodiments, the predetermined range of ferritin level during the treatment period is greater than or equal to 90 ng/mL and less than or equal to 650 ng/mL.

In some embodiments, the predetermined range of ferritin level during the treatment period is greater than or equal to 650 ng/mL.

In some embodiments, the JAK inhibitor is chosen from momelotinib or a pharmaceutically acceptable salt thereof, ruxolitinib, pacritinib or fedratinib. In some embodiments, the JAK inhibitor is momelotinib or a pharmaceutically acceptable salt thereof or ruxolitinib. In some embodiments, the JAK inhibitor is ruxolitinib. In some embodiments, the JAK inhibitor is pacritinib. In some embodiments, the JAK inhibitor is fedratinib. In some embodiments, the JAK inhibitor is momelotinib or a pharmaceutically acceptable salt thereof.

In some embodiments, the second therapeutic agent is a BET protein inhibitor, for example a BRD4 inhibitor. In some embodiments, the BET protein inhibitor is selected from GSK2820151, GSK525762, GS-5829, R06870810 (IV), BAY1238097, CC-90010, BMS-986158, 1NCB054329, 1NCB057643, ODM-207, AZD5153, FT-1101, ABBV-744, ABBV-075, PLX51107, B1894999, OTX015/MK8628, ZEN003694, RVX-000222, CPI-0610, apabetalone and fedratinib.

The treatment period during which the therapeutically effective stable dose is administered can be an extended period of time. In some embodiments, the plurality of weeks without dose reduction is 8 weeks or more, such as 10 weeks or more. In some embodiments, the plurality of weeks without dose reduction is 12 weeks or more. In some embodiments, the plurality of weeks without dose reduction is 24 weeks or more (e.g., more than 24 weeks), such as 28 weeks or more, 32 weeks or more, 36 weeks or more, 40 weeks or more, 44 weeks or more, 48 weeks or more, 52 weeks or more, or even more. In some embodiments, the treatment period without dose reduction is 1 year or more, such as 2 years or more, 3 years or more, 4 years or more, 5 years or more, 6 years or more, 7 years or more, or 8 years or more.

In some embodiments, the momelotinib or a pharmaceutically acceptable salt thereof is administered for a treatment period of a plurality of weeks. In some embodiments, the treatment period of a plurality of weeks is 12 weeks or more. In some embodiments, the treatment period of a plurality of weeks is 24 weeks or more. In some embodiments, the treatment period of a plurality of weeks is 36 weeks or more.

The subject methods can be utilized as a first line treatment for myelofibrosis. The patient to be treated according to the methods of this disclosure can be naive to Janus kinase inhibitor (JAKi) therapy. Second line treatment methods are also provided. In certain embodiments, the patient to be treated according to the methods of this disclosure has previously been treated with a JAK inhibitor. In particular embodiments, the JAK inhibitor is ruxolitinib (RUX). In particular embodiments, the JAK inhibitor is fedratinib. In certain cases, the previously-treated patient had an inadequate response to, or not deriving sufficient benefit from, or was intolerant of a Janus kinase inhibitor, e.g., RUX or fedratinib. In some embodiments, a patient who failed to respond or ceased to respond to previous therapy (e.g., with RUX or fedratinib) is treated according to the methods of this disclosure. In some case, the subject did not obtain any beneficial or desired clinical results from a prior treatment, e.g., as determined via a primary or secondary endpoint.

In certain embodiments, the subject or patient is one who (i) has not received any treatment (i.e. naive) for the disease, (ii) has received a prior treatment (e.g., JAKi, such as RUX or fedratinib) and is intolerant of the prior treatment; or (iii) is not deriving sufficient benefit from, did not respond or is resistant to, or is relapsed to a prior treatment (e.g., JAKi, such as RUX). In particular embodiments, the patient is not deriving sufficient benefit from a prior treatment (e.g., JAKi, such as RUX or fedratinib) because necessary dose reductions (e.g., due to an adverse event) result in less therapeutic benefit.

The treatment of ongoing disease, where the treatment stabilizes or reduces the undesirable clinical symptoms of the patient, is of particular interest. The expected progression-free survival times can be measured in months to years, depending on prognostic factors including the number of relapses, stage of disease, and other factors. Prolonging survival includes without limitation times of at least 1 month, about at least 2 months, about at least 3 months, about at least 4 months, about at least 6 months, about at least 1 year, about at least 2 years, about at least 3 years, or more. Overall survival can also be measured in months to years. The patient's symptoms may remain static or may decrease.

The compounds of the present application or the compositions thereof may be administered once, twice, three, or four times daily, using any suitable mode described herein. Also, administration or treatment with MMB may be continued for a number of days; for example, commonly treatment would continue for at least 7 days, 14 days, or 28 days, for one cycle of treatment. Treatment cycles are generally known and are frequently alternated with resting periods of about 1 to 28 days, commonly about 7 days or about 14 days, between cycles. The treatment cycles, in other embodiments, may also be continuous.

In the treatment of an identified subject, an appropriate unit dose of the MMB compound will generally be about 0.01 to 500 mg per kg patient body weight per day which can be administered in single or multiple doses. The dosage level will be about 0.1 to about 250 mg/kg per day; such as about 0.5 to about 100 mg/kg per day. A suitable dosage level may be about 0.01 to 250 mg/kg per day, about 0.05 to 100 mg/kg per day, or about 0.1 to 50 mg/kg per day. Within this range the dosage may be 0.05 to 0.5, 0.5 to 5 or 5 to 50 mg/kg per day. Suitable unit doses will typically be in the range from 10 to 500 mg, such as 50-400 mg. e.g., 100, 150, 200, 250 or 300 mg. For oral administration, the compositions are preferably provided in the form of tablets containing 1.0 to 1000 milligrams of the active ingredient, particularly 1, 5, 10, 15, 20, 25, 50, 75, 100, 150, 200, 250, 300, 400, 500, 600, 750, 800, 900, and 1000 milligrams of the active ingredient. The dosage may be selected, for example to any dose within any of these ranges, for therapeutic efficacy and/or symptomatic adjustment of the dosage to the patient to be treated. The compound will preferably be administered on a regimen of 1 to 4 times per day, preferably once or twice per day.

It will be understood that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

In some embodiments of the subject methods, the therapeutic effective amount of the MMB compound described herein is a dose of 50 mg, 100 mg. 150 mg, 200 mg, 250 mg, or 300 mg. In certain cases, the therapeutically effective amount is between 100 mg/day and 300 mg/day, such as 100 mg/day, 150 mg/day, 200 mg/day, 250 mg/day, or 300 mg/day. In certain cases, the therapeutically effective amount is between 50 mg/day and 200 mg/day, such as 50 mg/day, 100 mg/day, 150 mg/day, or 200 mg/day. In some embodiments of the subject methods, the therapeutic effective amount is 200 mg/day. The administration can be oral. In some cases, the administration is once daily. In some cases, the administration is BID, e.g., in equally divided doses. In certain cases, the MMB is administered with food. In certain cases, the MMB is administered without food.

For patients unable to ingest tablets orally, the therapeutic effective amount of the MMB compound can be administered through another route, e.g., via a nasogastric tube.

In some embodiments, the improved outcome from MMB therapy that results from practicing the subject methods can be manifested as maintaining transfusion independence. In some embodiments, the improved outcome from MMB therapy that results from practicing the subject methods can be manifested as achieving transfusion independence. In some embodiments, the improved outcome from MMB therapy that results from practicing the subject methods can be manifested as maintaining or achieving WK24 TI-R. In some embodiments, the improved outcome from MMB therapy that results from practicing the subject methods can be manifested as an improvement in one or more clinical endpoints, such as anemia response, in spleen response and/or symptom response. In some embodiments, the improved outcome from MMB therapy that results from practicing the subject methods can be manifested as prolonging overall survival for, for example, of at least 1 month, about at least 2 months, about at least 3 months, about at least 4 months, about at least 6 months, about at least 1 year, about at least 2 years, about at least 3 years, or more.

### VI. Momelotinib

In the methods described herein, momelotinib, or a pharmaceutically acceptable salt thereof, is administered in therapeutically effective amounts.

Momelotinib (MMB) is an inhibitor of JAK (JAK1 and JAK2) and ACVR1 that is also known as N-(cyanomethyl)-4-(2-(4-morpholinophenylamino)pyrimidin-4-yl)benzamide or CYT-0387 and has the structure of Structure I:

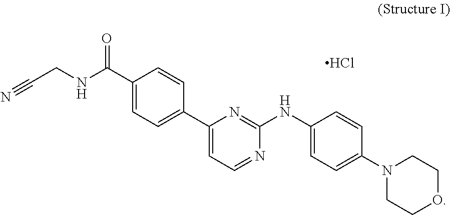

“Pharmaceutically acceptable salt” refers to a salt of a compound that retains the biological effectiveness and properties of the underlying compound, and which is not biologically or otherwise undesirable. MMB can be present as an acid addition salt. Pharmaceutically acceptable acid addition salts of basic drugs may be prepared using inorganic and organic acids. Acids useful for reaction with MMB to form pharmaceutically acceptable salts (acid addition salts), and methods for accomplishing same, are known to skilled artisans. If the MMB is present as an acid addition salt, MMB free base can be obtained by basifying a solution of the acid salt. A solvate is formed by the interaction of a solvent and a MMB compound. Solvates of salts of the MMB compounds described herein are also used in particular embodiments of the methods described herein. In some cases, the MMB compound solvate is a hydrate.

Embodiments of the methods disclosed herein use any convenient acid addition pharmaceutically acceptable salts of momelotinib, or a solvate or hydrate thereof. In some embodiments, the momelotinib compound is a hydrochloride salt. In certain embodiments, the compound hydrochloride salt that finds use in the subject methods is momelotinib monohydrochloride of Structure II:

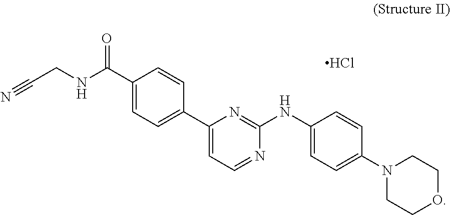

In certain embodiments, the momelotinib monohydrochloride salt is anhydrous. In certain embodiments, the momelotinib monohydrochloride salt is a hydrate, e.g., a monohydrate.

In particular embodiments, the momelotinib hydrochloride salt used is momelotinib dihydrochloride of Structure III:

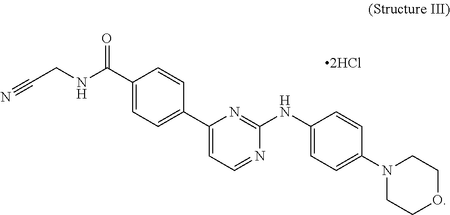

In certain embodiments, the momelotinib dihydrochloride salt is anhydrous. In certain embodiments, the momelotinib dihydrochloride salt is a hydrate, e.g., momelotinib dihydrochloride monohydrate.

Embodiments of the methods described herein use any crystalline salt forms of momelotinib, including but not limited to, those forms described in WO2015191846, the disclosure of which is herein incorporated by reference. In some embodiments, a crystalline salt form of momelotinib is referred to as a polymorph form of the compound.

In some embodiments, the MMB compound used is momelotinib dihydrochloride monohydrate that is in crystalline Form II. The crystalline Form II is characterized by one or more parameters, as follows. The crystalline Form II can be characterized by crystals with unit cell parameters at T=100° K of: a=10.2837(6) A, b=10.4981(6) A, c=11.5143(7) A, a=83.297(2°), β=87.649(2°), γ=67.445(2°), and a triclinic P-1 space group. The crystalline Form II can be characterized by an x-ray powder diffraction (XRPD) pattern having peaks at 7.7°, 19.3°, 24.0°, 25.7°, and 29.6° 2-Θ+0.2° 2-Θ.

In some embodiments, the MMB compound used is momelotinib monohydrochloride anhydrous that is in crystalline Form I. The crystalline Form I is characterized by one or more parameters, as follows. The crystalline Form I can be characterized by an X-ray powder diffraction (“XRPD”) pattern having peaks at 13.5°, 20.9°, 26.1°, 26.6°, and 28.3° 2-Θ+0.2° 2-Θ.

In some embodiments, the MMB compound used is momelotinib monohydrochloride anhydrous that is in crystalline Form III. The crystalline Form III is characterized by one or more parameters, as follows. The crystalline Form III can be characterized by an X-ray powder diffraction pattern having peaks at 12.7°, 14.6°, 17.8°, 19.7° and 23.3° 2-Θ+0.2° 2-Θ.

In some embodiments, the MMB compound used is momelotinib dihydrochloride anhydrous Form IV. The crystalline Form IV is characterized by one or more parameters, as follows. The crystalline Form IV can have an XRPD pattern having peaks at 5.5°, 10.1°, 14.9°, 25.1°, and 26.6° 2-Θ+0.2° 2-Θ.

TABLE 1
XRPD peaks for momelotinib forms

| MMB | | MMB | | MMB | | MMB | |
|---|---|---|---|---|---|---|---|
| dihydrochloride | | dihydrochloride | | monohydrochloride | | monohydrochloride | |
| anhydrous | | monohydrate | | anhydrous | | anhydrous | |
| Form IV | | Form II | | Form I | | Form III | |
| | Relative | | Relative | | Relative | | Relative |
| Position | Intensity | Position | Intensity | Position | Intensity | Position | Intensity |
| [°2th.] | [%] | [°2th.] | [%] | [°2th.] | [%] | [°2th.] | [%] |
| 5.5 | 31.0 | 7.7 | 33.7 | 13.5 | 15.3 | 12.7 | 85.0 |
| 10.1 | 100.0 | 19.3 | 43.7 | 20.9 | 100.0 | 14.6 | 50.0 |
| 14.9 | 66.5 | 24.0 | 100.0 | 26.1 | 20.6 | 17.8 | 55.5 |
| 25.1 | 86.7 | 25.7 | 79.0 | 26.6 | 15.5 | 19.7 | 100.0 |
| 26.6 | 69.3 | 29.6 | 35.7 | 28.3 | 16.6 | 23.3 | 60.1 |

The following patent applications are incorporated by reference for all purposes, including but not limited to the use of MMB described therein: International application no. PCT/AU2008/000339, filed on Mar. 12, 2008; and International application no. PCT/AU2011/001551, filed on Nov. 29, 2011; and International application no. PCT/US2015/035316, filed on Jun. 11, 2015; and International application no.

PCT/US2017/045957, filed on Aug. 8, 2017.

### VII. Pharmaceutical Compositions

The MMB compounds are usually administered in the form of pharmaceutical compositions. Embodiments of the methods disclosed herein include administering a pharmaceutical composition that contains a MMB compound disclosed herein or a pharmaceutically acceptable salt, or solvate or hydrate thereof, and one or more pharmaceutically acceptable vehicles selected from carriers, adjuvants and excipients.

The pharmaceutical compositions may be administered in either single or multiple doses. The pharmaceutical composition may be administered by various methods. In certain embodiments, the pharmaceutical composition is administered by intra-arterial injection, intravenously, intraperitoneally, parenterally, intramuscularly, subcutaneously, orally, topically, or as an inhalant.

Oral administration is a currently preferred route for administration of the MMB compounds described herein. In currently preferred embodiments, the forms or compositions of MMB thereof described herein are formulated for oral administration using pharmaceutically acceptable carriers. Pharmaceutical compositions formulated for oral administration can be in the form of tablets, capsules, cachets, dragees, lozenges, liquids, gels, syrups, slurries, elixirs, suspensions, or powders.

Administration may be via, for example, capsule or enteric coated tablets. In making the pharmaceutical compositions that include a MMB compound described herein or a pharmaceutically acceptable salt, or solvate or hydrate thereof, the active ingredient is usually diluted by an excipient and/or enclosed within such a carrier that can be in the form of a capsule, sachet, paper or other container. When the excipient serves as a diluent, it can be in the form of a solid, semi-solid, or liquid material, which acts as a vehicle, carrier or medium for the active ingredient. Thus, the compositions can be in the form of tablets, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders.

For preparing solid compositions such as tablets, the principal active ingredient may be mixed with a pharmaceutical excipient to form a solid preformulation composition containing a homogeneous mixture of a compound of any of the above formulae or a pharmaceutically acceptable salt, prodrug, or solvate thereof. When referring to these preformulation compositions as homogeneous, the active ingredient may be dispersed evenly throughout the composition so that the composition may be readily subdivided into equally effective unit dosage forms such as tablets, and capsules. The tablets of the MMB compounds described herein may be coated or otherwise compounded to provide a dosage form affording the advantage of prolonged action, or to protect from the acid conditions of the stomach. For example, the tablet or pill can include an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The specific dose level of a MMB compound described herein for any particular subject will depend upon a variety of factors including the activity of the specific compound employed, the age, body weight, general health, sex, diet, time of administration, route of administration, and rate of excretion, drug combination and the severity of the particular disease in the subject undergoing therapy. For example, a dosage may be expressed as a number of milligrams of a MMB compound per kilogram of the subject's body weight (mg/kg). Dosages of between about 0.01 and 200 mg/kg may be appropriate. In some embodiments, about 0.01 and 150 mg/kg may be appropriate. In other embodiments a dosage of between 0.05 and 100 mg/kg may be appropriate. Normalizing according to the subject's body weight is particularly useful when adjusting dosages between subjects of widely disparate size, such as occurs when using the drug in both children and adult humans or when converting an effective dosage in a non-human subject such as dog to a dosage suitable for a human subject.

### VIII. Pharmaceutically Acceptable Vehicles

Pharmaceutically acceptable vehicles include carriers, adjuvants and excipients, for example, inert solid diluents and fillers, diluents, including sterile aqueous solution and various organic solvents, permeation enhancers, solubilizers and adjuvants.

The term “carrier” refers to diluents or fillers, disintegrants, precipitation inhibitors, surfactants, glidants, binders, lubricants, anti-oxidants, and other excipients and vehicles with which the MMB compound is administered. Examples of carriers that are useful in dosage forms administered in the methods described herein include, but are not limited to, aluminum monostearate, aluminum stearate, carboxymethylcellulose, carboxymethylcellulose sodium, crospovidone, glyceryl isostearate, glyceryl monostearate, hydroxyethyl cellulose, hydroxyethyl cellulose, hydroxymethyl cellulose, hydroxyoctacosanyl hydroxystearate, hydroxypropyl cellulose, hydroxypropyl cellulose, hydroxypropyl methylcellulose, lactose, lactose monohydrate, magnesium stearate, mannitol, microcrystalline cellulose, poloxamer 124, poloxamer 181, poloxamer 182, poloxamer 188, poloxamer 237, poloxamer 407, povidone, silicon dioxide, colloidal silicon dioxide, silicone, silicone adhesive 4102, and silicone emulsion. It should be understood, however, that the carriers selected for the pharmaceutical compositions provided in the present disclosure, and the amounts of such carriers in the composition, may vary depending on the method of formulation (e.g., dry granulation formulation, solid dispersion formulation).

The term “diluent” or “filler” generally refers to a substance that is used to dilute the MMB compound prior to delivery. Diluents can also serve to stabilize compounds. Examples of diluents may include starch, saccharides, disaccharides, sucrose, lactose, polysaccharides, cellulose, cellulose ethers, hydroxypropyl cellulose, sugar alcohols, xylitol, sorbitol, maltitol, microcrystalline cellulose, calcium or sodium carbonate, lactose, lactose monohydrate, dicalcium phosphate, cellulose, compressible sugars, dibasic calcium phosphate dehydrate, mannitol, microcrystalline cellulose, and tribasic calcium phosphate.

The term “disintegrant” generally refers to a substance which, upon addition to a solid preparation, facilitates its break-up or disintegration after administration and permits the release of an active ingredient as efficiently as possible to allow for its rapid dissolution. Examples of disintegrants include maize starch, sodium starch glycolate, croscarmellose sodium, crospovidone, microcrystalline cellulose, modified corn starch, sodium carboxymethyl starch, povidone, pregelatinized starch, and alginic acid.

The term “precipitation inhibitors” generally refers to a substance that prevents or inhibits precipitation of the active agent. One example of a precipitation inhibitor includes hydroxypropylmethylcellulose.

The term “surfactants” generally refers to compounds that lower the surface tension between two liquids or between a liquid and a solid. Examples of surfactants include poloxamer and sodium lauryl sulfate.

The term “glidant” generally refers to a substance used in tablet and capsule formulations to improve flow-properties during tablet compression and to produce an anti-caking effect. Examples of glidants include colloidal silicon dioxide, talc, fumed silica, starch, starch derivatives, and bentonite.

The term “binder” generally refers to any pharmaceutically acceptable film which can be used to bind together the active and inert components of the carrier together to maintain cohesive and discrete portions. Examples of binders include hydroxypropylcellulose, hydroxypropylmethylcellulose, povidone, copovidone, ethyl cellulose, gelatin, and polyethylene glycol.

The term “lubricant” generally refers to a substance that is added to a powder blend to prevent the compacted powder mass from sticking to the equipment during the tableting or encapsulation process. A lubricant can aid the ejection of the tablet from the dies during tableting, and can improve powder flow. Examples of lubricants include magnesium stearate, stearic acid, silica, fats, calcium stearate, polyethylene glycol, sodium stearyl fumarate, or talc; and solubilizers such as fatty acids including lauric acid, oleic acid, and Cg/Cio fatty acid.

The term “anti-oxidant” generally refers to a substance that inhibits the oxidation of other substances. In certain embodiments of the invention, anti-oxidants are added to the pharmaceutical composition. Examples of anti-oxidants include ethylenediaminetetraacetic acid, ethylenediaminetetraacetic acid disodium salt, sodium sulfite, sodium metabisulfite, sodium bisulfite, butylated hydroxytoluene (BHT), butylated hydroxyanisole (BHA), ascorbic acid, ascorbyl palmitate, thioglycerol, thioglycolic acid, tocopherol (vitamin E), D-a tocopheryl polyethylene glycol 1000 succinate (vitamin E TPGS) and propyl gallate. In certain embodiments, the antioxidant is propyl gallate.

In some embodiments, the pharmaceutical composition includes MMB, for example MMB dihydrochloride monohydrate Form II, and an antioxidant selected from butylated hydroxyanisole (BHA), ascorbic acid, and the antioxidant propyl gallate. The antioxidant may be present in an amount sufficient to prevent, inhibit, and/or reduce degradation of the MMB active ingredient (such as MMB Form II). By way of examples, the antioxidant may be present in an amount of about 0.001, about 0.002%, about 0.005%, about 0.01%, about 0.02%, about 0.05%, about 0.1%, about 0.2%, about 0.5%, or about 1% by weight in the pharmaceutical composition. In one embodiment, the pharmaceutical composition includes propyl gallate at an amount of about 0.001%, about 0.01%, about 0.1%, about 0.2%, about 0.5%, or about 1%. In particular embodiments, the pharmaceutical composition includes MMB, for example MMB dihydrochloride monohydrate Form II, and about 0.2% of propyl gallate.

In some embodiments, the pharmaceutical composition includes at least one or at least two diluent(s). In certain embodiments, the pharmaceutical composition includes one or two diluent(s). In particular embodiments, the diluent is selected from mannitol, microcrystalline cellulose, lactose, dextrose, sucrose, ludiflash, F-melt, advantose, GalenlQ, and any mixtures thereof. In one embodiment, the diluent is mannitol, microcrystalline cellulose, or a mixture thereof.

In some embodiments, the pharmaceutical composition includes at least one disintegrant. In certain embodiments, the pharmaceutical composition includes one disintegrant. In a particular embodiment, the disintegrant is sodium starch glycolate. In one embodiment, the disintegrant is croscarmellose sodium. In another embodiment, the disintegrant is crospovidone.

In some embodiments, the pharmaceutical composition includes at least one glidant. In certain embodiments, the pharmaceutical composition includes one glidant. In one embodiment, the glidant is colloidal silicon dioxide.

In some embodiments, the pharmaceutical composition includes at least one lubricant. In certain embodiments, the pharmaceutical composition includes one lubricant. In one embodiment, the lubricant is magnesium stearate.

It should be understood that the pharmaceutical composition includes pharmaceutically acceptable carriers detailed herein, the same as if each and every combination of pharmaceutically acceptable carrier were specifically and individually listed.

### IX. Unit Dosage Forms

In some embodiments, the pharmaceutical compositions as described herein are formulated in a unit dosage form. The term “unit dosage forms” refers to physically discrete units suitable as unitary dosages for subjects (e.g., human subjects and other mammals), each unit containing a predetermined quantity of MMB compound active material calculated to produce the desired therapeutic effect, in association with a suitable pharmaceutical carrier. In certain embodiments of the invention, the unit dosage form includes at least one pharmaceutically acceptable carrier.

In a further embodiment, the unit dosage forms include any one of the forms of MMB, for example MMB dihydrochloride monohydrate Form II. In some embodiments, the unit dosage form includes any one of the forms of MMB, for example MMB dihydrochloride monohydrate Form II, in amount equivalent to form about 10 mg to about 1000 mg, about 10 mg to about 800 mg, about 10 mg to about 700 mg about 10 mg to about 500 mg, about 10 mg to about 400 mg, about 10 mg to about 300 mg, about 10 mg to about 250 mg, about 10 mg to about 200 mg, about 10 mg to about 150 mg, about 10 mg to about 100 mg, about 10 mg to about 50 mg, about 50 mg to about 1000 mg, about 50 mg to about 800 mg, about 50 mg to about 700 mg about 50 mg to about 500 mg, about 50 mg to about 400 mg, about 50 mg to about 300 mg, about 50 mg to about 250 mg, about 50 mg to about 200 mg, about 50 mg to about 150 mg, about 50 mg to about 100 mg, about 100 mg to about 1000 mgs, about 100 mg to about 800 mg, about 100 mg to about 700 mg about 100 mg to about 500 mg, about 100 mg to about 400 mg, about 100 mg to about 300 mg, about 100 mg to about 250 mg, about 100 mg to about 200 mg, about 150 mg to about 300 mg, about 150 mg to about 250 mg, about 150 mg to about 200 mg, about 200 mg to about 300 mg, about 200 mg to about 250 mg, or about 200 mg to about 300 mg of MMB free base.

The pharmaceutical compositions described herein can be manufactured using any conventional method, such as, but not limited to, mixing, dissolving, granulating, dragee-making, levigating, emulsifying, encapsulating, entrapping, melt-spinning, spray-drying, or lyophilizing processes. A skilled artisan would recognize suitable methods and techniques to prepare a tablet by conventional formulation. Exemplary methods and techniques to prepare powders for compression into a tablet include dry granulation or wet granulation. Dry granulation generally refers to the process of forming granules without using a liquid solution, whereas wet granulation generally refers to the process of adding a liquid solution to powders to granulate.

### X. Ruxolitinib Therapy

Aspects of this disclosure include methods of treating a subject for a myclofibrosis using MMB as a second line therapy. In some embodiments, the subject has been treated with a previous first line JAK inhibitor therapy, such as ruxolitinib. Further details of conventional ruxolitinib therapy of the SIMPLIFY 1 and 2 clinical trials can be found in publicly accessible trial protocols.

Ruxolitinib is typically administrated BID in equally divided doses. The range of recommended doses for patients with myelofibrosis is 10-fold, from a high of 25 mg twice daily to a low of 5 mg once daily. The recommended starting dose of ruxolitinib is based on platelet count, with an attenuated starting dose of 15 mg twice daily recommended for patients whose pre-treatment platelet count is 100 to 200×10⁹/L. A complete blood count (CBC) and platelet count is performed before initiating therapy, every 2 to 4 weeks until doses are stabilized, and then as clinically indicated. Doses may be titrated based on safety and efficacy. Ruxolitinib dose modification guidelines in response to symptom or disease progression are also available in publicly accessible trial protocols and reports.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### EXAMPLES

Below are examples of specific embodiments for carrying out the present invention, the examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

### Example 1. Week 24 Response and Overall Survival

The relationship between W24 response and overall survival (OS) was explored in patients randomized to MMB in the SIMPLFY-1 (S1) and SIMPLIFY-2 (S2) trials.

Responders were defined as follows:
W24 TI response (TI-R): no RBC transfusion within ?12 weeks immediately prior to W24, with Hgb≥8 g/dL
W24 spleen response: ≥35% spleen volume reduction vs baseline (BL)
W24 symptom response: ≥50% reduction in MFSAF total symptom score vs BL

Survival from baseline was estimated using K-M analysis with descriptive log-rank tests for comparison applied (all p values are descriptive). Hazard ratios were computed using proportional hazard regression. To adjust for the time to response bias, overall survival from W24 was also compared. As patients randomized to the control arm in both studies and crossed over to open label MMB after 24 weeks of randomized treatment, a valid comparison of overall survival with patients randomized to RUX or BAT/RUX vs MMB was not possible, therefore, only the MMB arm was analyzed.

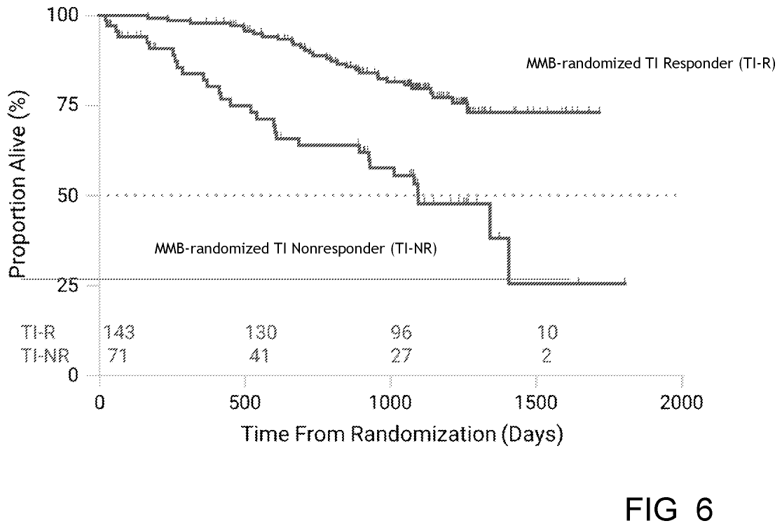

In S1 the overall survival in all patients randomized to MMB by Week 24 TI Response is shown in FIG. 6. The patients randomized to MMB who were TI responders at W24 show an overall survival advantage. The 3-year survival in MMB TI responders was 80% compared to 50% in MMB TI nonresponders, HR=0.30; p<0.0001.

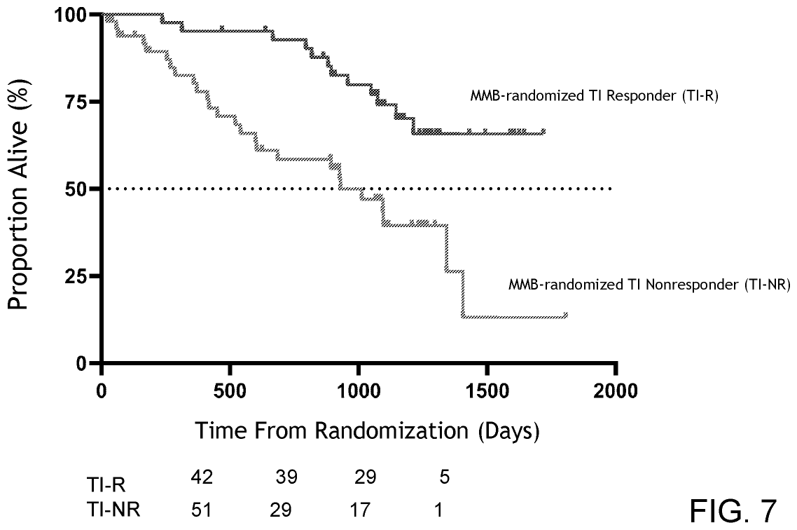

In S1 the overall survival in anemic patients randomized to MMB by Week 24 TI Response is shown in FIG. 7. Week 24 TI response rates in patients with baseline anemia receiving MMB was 45% and receiving RUX was 26%. Consistent with results in the total MMB population, MMB also shows a significant overall survival advantage in S1 patients with anemia at baseline (RBC transfusion ≤12 weeks before baseline, or baseline Hgb<10 g/dL) who achieve a TI-R at Week 24, HR=0.32; p=0.0006.

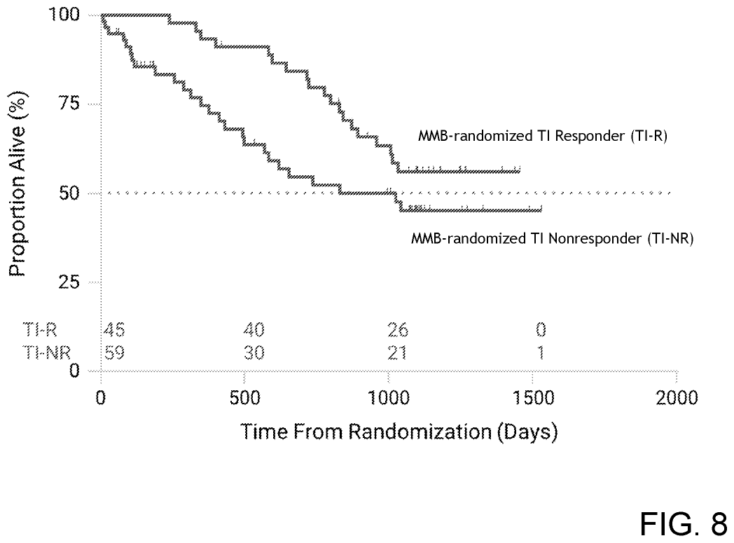

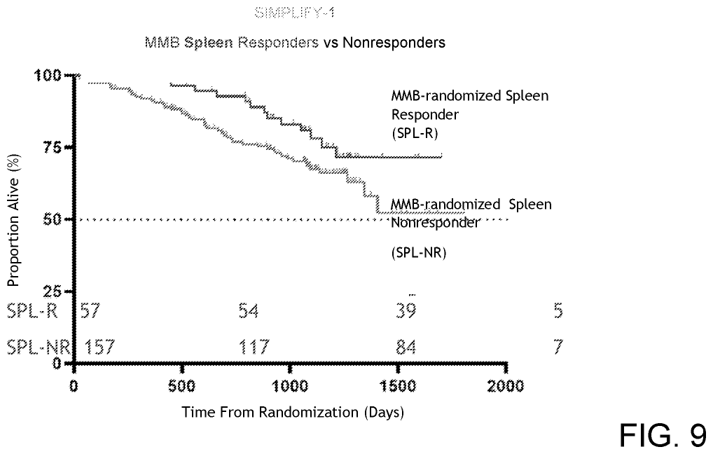

In S2 the overall survival in all patients randomized to MMB by Week 24 TI Response is shown in FIG. 8. The patients randomized to MMB who were TI responders at W24 showed a trend toward better overall survival compared to TI non-responders, HR=0.57; p=0.0652.

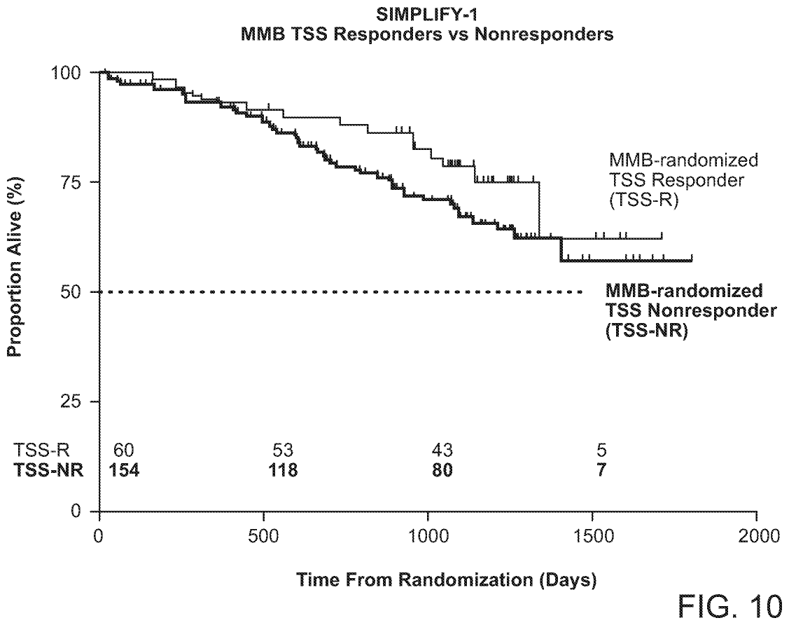

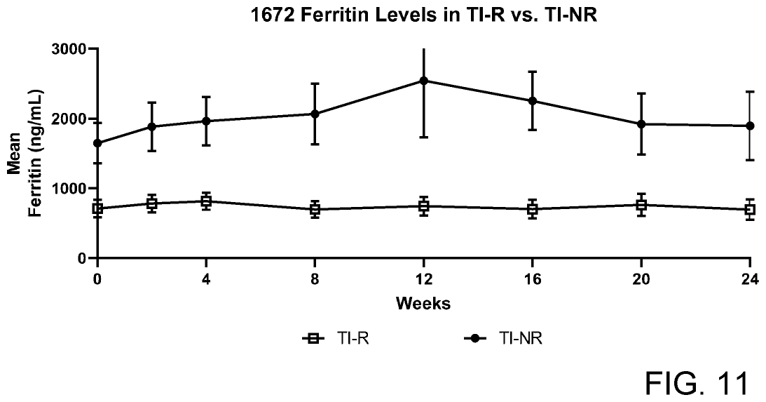

The relationship between W24 spleen and symptom responses and overall survival were also explored in MMB patients. FIGS. 9 and 10 show the overall survival in spleen and TSS responders, respectively from S1. In S1, MMB spleen responders showed an advantage in overall survival when compared to MMB nonresponders, HR=0.59; p=0.0904. Likewise, MMB symptoms (TSS) responders showed an advantage in overall survival when compared to MMB nonresponders, HR=0.66; p=0.1657. In S2, overall survival in MMB symptom (TSS) responders compared to MMB TSS nonresponders showed HR=0.67 and p=0.2513 (data not shown).

Momelotinib (MMB) provided anemia benefit via inhibition of JAK1, JAK2 and ACVR1/ALK2, resulting in higher rates of transfusion independence (TI) compared with ruxolitinib (RUX), regardless of degree of baseline anemia, or the baseline platelet count or transfusion status. In additions, myclofibrosis patients that received MMB converted to, or maintained TI with significantly improved overall survival (OS).

Week 24 (W24) TI-Response was a strong predictor of improved survival in both the SIMPLIFY-1 and SIMPLIFY-2 trials. This analysis showed that W24 TI-Response was a predictor of improved survival in MMB patients who were anemic at baseline. It also showed that the correlation between W24 TI-Response and overall survival observed with MMB supports the clinical relevance of TI in MF patients receiving MMB.

These results show that likelihood of achieving W24 TI-Response should be a consideration regarding the choice of treatment.

### Example 2. Ferritin as a Biomarker for Week 24 Transfusion Response

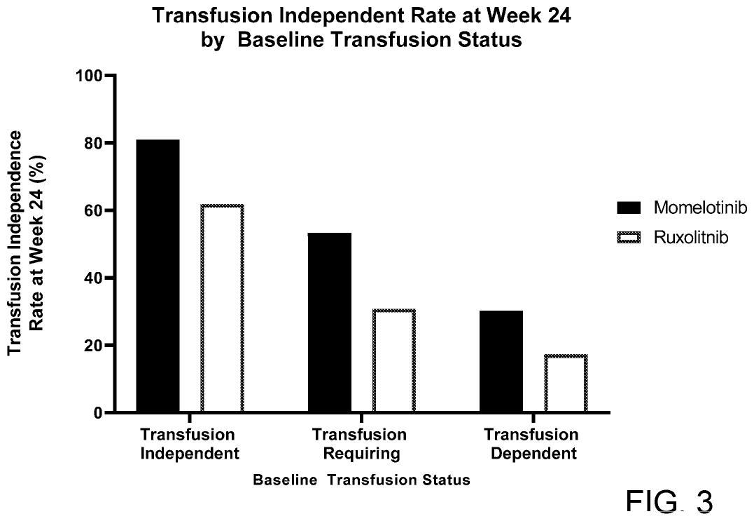

FIG. 3 shows the degree that the baseline transfusion requirement influences W24 TI-R. Although MMB was better than RUX for each baseline TI/TR/TD cohort, the more transfusions a patient needed at baseline, the lower the chance of the patient becoming a W24 TI-responder.

A previous study (the “1672 Study”) has shown that highly inflamed patients were unable to achieve transfusion independence from MMB alone. Elevated serum ferritin, C-reactive protein (CRP), hepcidin and WBCs are commonly associated with significant inflammation and importantly are easily measured in the clinic. [Oh et al. 2020 Blood Adv. 4(18) pp. 42824291]. This study showed that momelotinib reversed or reduced transfusion dependency in transfusion-dependent myelofibrosis patients and inhibited hepcidin. The 1672 Study showed stabilization of ferritin in TI-R patients over 24-weeks of treatment. Like hepcidin, baseline ferritin is a strong negative prognostic factor in patients with MF. FIG. 11 shows mean ferritin levels in TI-R and TI-NR patients over 24 weeks in the 1672 study.

As demonstrated below baseline and on-treatment ferritin level thresholds can be used to predict MMB TI-R.

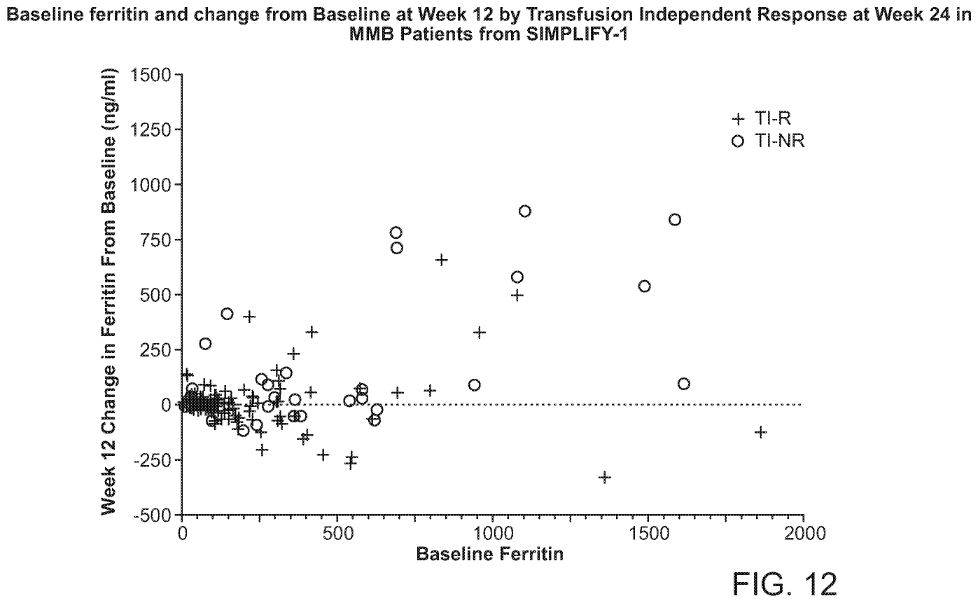

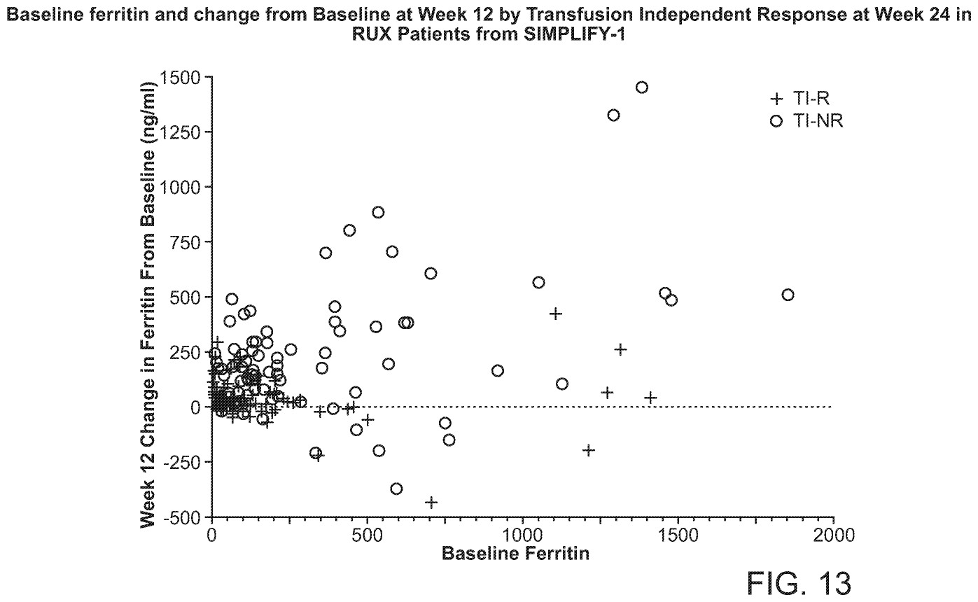

In S1, a lower level of baseline ferritin in baseline TI (˜150 ng/mL) was observed compared to baseline-Non-TI subjects (>800 ng/mL), consistent with ferritin's role in the anemia of MF patients. MMB was more effective at stabilizing ferritin over time when compared to RUX. FIGS. 12 and 13 show that when compared to RUX treated patients in S1, ferritin levels in MMB treated patients in S1 hold stable, with a very modest change in most TI-R patients by Week 12. RUX treated patients that have greater than 100 ng/ml increase in ferritin by week 12 were associated with TI-NR at Week 24.

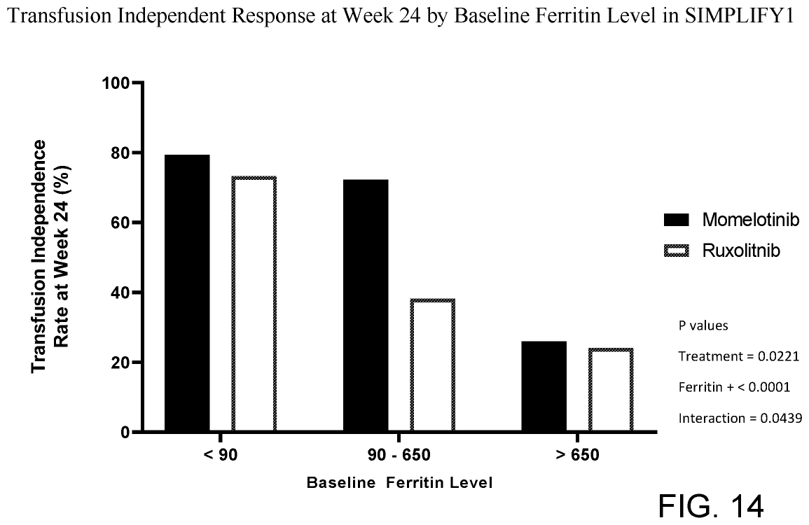

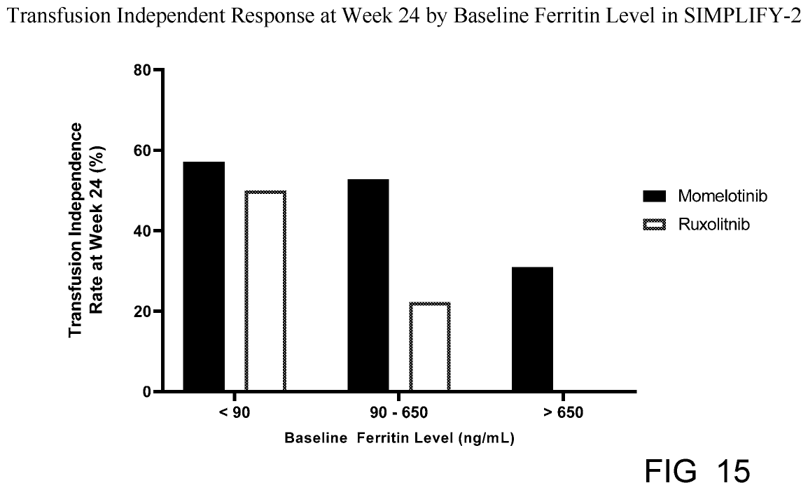

FIG. 14 shows that the baseline ferritin levels in JAKi naïve patients of S1. A baseline ferritin level of less than 650 ng/mL correlates with MMB W24 TI-R. MMB was superior to RUX for each ferritin cohort and W24 TI-R was dramatically higher in the 90 ng/mL-650 ng/mL cohort. RUX was much less effective in patients above 90 ng/mL. Although MMB overall activity drops when a patient's ferritin levels exceed 650 ng/mL, MMB was still nominally better than RUX in patient's whose ferritin levels exceed 650 ng/mL. Less than 12% of the JAKi naïve population have ferritin levels above 650 ng/mL. FIG. 15 shows that the baseline ferritin levels in RUX patients in S2. The same trend is seen in this study when these thresholds were applied to the more advanced SIMPLIFY-2 population, demonstrating that ferritin can be a predictive biomarker for MMB W24 TI-R. MMB treated patients presenting with ferritin levels less than 650 ng/ml have a higher probability of becoming week 24 TI-R. Slightly higher TI-R rate in the greater than 650 ng/ml cohort for this study may be a consequence of switching from RUX. It appears that inflated ferritin levels caused by RUX treatment can be reversed when treatment is switched from RUX to MMB.

### Example 3. Ferritin as a Biomarker for Week 24 Transfusion Response

Myelofibrosis (MF) typically presents with constitutional symptoms, splenomegaly, and anemia with the degree of anemia and transfusion dependency being among the most important predictors of poor overall survival (OS). Momelotinib (MMB) is a differentiated JAKi. JAK2 inhibitor with potent activity against ACVR1/ALK2, a critical regulator of hepcidin production and iron metabolism.

MMB has demonstrated robust clinical activity against all 3 of these hallmark features of MF in the SIMPLIFY Ph 3 studies including SIMPLIFY-1 (S1) in the JAK inhibitor (JAKi) naïve setting when compared directly to ruxolitinib (RUX) and in the previously JAKi experienced patients (SIMPLIFY-2 [S2]) in comparison to best available therapy (BAT). MMB has also demonstrated robust OS in JAKi naïve population (S1) and JAKi experienced population (S2) (Verstovsek et. al. 2020). Critically, patients randomized to MMB who achieve or maintain transfusion independence (TI) by Week 24 (W24) had better OS, further suggesting that MMB's anemia benefits positively impact long term outcome in patients with MF (Mesa et al. EHA Conference 2021).

Increased hepcidin and ferritin are associated with dysregulated iron metabolism and inflammation, both of which have previously been shown to be strong negative prognostic factors for OS in patients with MF at the time of first referral (Pardanani et al. 2013). A Phase 2 translational biology study previously demonstrated MMB acutely and chronically suppresses elevated levels of hepcidin and restores iron homeostasis in transfusion dependent patients who achieve a W24 TI-response (Oh et al. 2020 Blood Adv. 4(18) pp. 4282-4291).

### Methods

Based on the findings from the Phase 2 translational biology study we retrospectively analyzed the relationship between serum ferritin, hepcidin and C-reactive protein (CRP) and the W24 TI response rates for patients randomized to MMB and RUX in the S1 study using univariate analyses and multiple logistic regression models. These findings were then independently confirmed in the previously RUX treated patients in S2.

### Results

These analyses identified pre-treatment serum ferritin level as having the greatest predictive interaction between MMB and RUX treatment effect on W24 TI-R rate in JAKi naïve patients from S1. TI-R treatment effect of MMB vs. RUX was significantly greater (p=0.0051 for the interaction) in the ≥90 ng/mL cohort (62% vs. 35% respectively) than in the <90 ng/mL cohort (79% and 73%). These findings were confirmed in S2 study of a smaller sample size, where TI-R treatment effect of MMB vs. BAT/RUX was greater in the ≥90 ng/mL cohort (41% vs. 11% respectively) than in the <90 ng/mL cohort (57% and 50%).

Data also demonstrated a significant increase in serum ferritin for patients randomized to RUX compared to MMB at W24 from baseline (RUX mean ferritin change +226.1 ng/mL vs MMB+13.8 ng/mL, p=0.0003), irrespective of baseline ferritin.

| | | | TI- | | | |
|---|---|---|---|---|---|---|
| | | | Response | | | |
| | Baseline | Sample | Rate at | Response Ratio | | P-Value for |
| Study | Ferritin | Size | Week 24 | (MMB/Control) | P-value | Interaction* |
| SIMPLIFY-1 | <90 ng/mL | MMB: 68 | MMB: 79% | 1.0843 | 0.3926 | 0.0051 |
| (JAKi Naïve) | | RUX: 71 | RUX: 73% | | | |
| | ≥90 ng/mL | MMB: 117 | MMB: 62% | 1.7538 | <0.0001 | |
| | | RUX: 114 | RUX: 35% | | | |
| SIMPLIFY-2 | <90 ng/mL | MMB: 14 | MMB: 57% | 1.1429 | 0.7463 | 0.0707 |
| (RUX- | | BAT: 8 | BAT: 50% | | | |
| Experienced) | ≥90 ng/mL | MMB: 78 | MMB: 41% | 3.7949 | <0.0001 | |
| | | BAT: 37 | BAT: 11% | | | |

*p-value for testing whether MMB treatment effect on TI response rate at week 24 vs control was the same across baseline ferritin groups.
Note:
response ratios, p-values for response ratios and p-values for interaction were from generalized linear models with log link with TI response at week 24 as the dependent variable and treatment, baseline ferritin and interaction between the two as covariates.

### CONCLUSION

Ferritin is a well-established and easily measured clinical biomarker that is associated with both iron metabolism and uncontrolled inflammation; however, it is not routinely measured in patients with myelofibrosis. Prior analyses demonstrate patients treated with MMB who achieve a W24 TI-R have increased OS compared to non-TI responders. These new analyses expand on these findings and strongly demonstrate MMB vs. RUX treatment effect is greater in baseline serum ferritin ≥90 ng/mL vs. <90 ng/mL as identified in JAKi naïve patients and independently confirmed in previously RUX-experienced patients. Further, data presented here show JAKi naïve patients from S1 randomized to RUX have significantly elevated ferritin levels by W24 when compared to MMB. In totality these data suggest serum ferritin may become an important biomarker to help inform therapy selection in the front line as well as potentially guide the transition to MMB in the post-RUX setting. This association should be examined prospectively in future MMB trials.

### EQUIVALENTS

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112(6) is not invoked.

### EMBODIMENTS OF THE INVENTION

1. A method of treating myelofibrosis in a subject, the method comprising:
administering a therapeutically effective amount of momelotinib or a pharmaceutically acceptable salt thereof to a subject identified as having (i) myelofibrosis, and (ii) a ferritin level greater than 90 ng/mL.
   2. The method of embodiment 1, wherein the subject is identified as having a ferritin level greater than 90 ng/mL and less than 650 ng/mL.
   3. The method of embodiment 1, wherein the subject is identified as having a ferritin level greater than 650 ng/mL.
   4. The method of embodiment 3, further comprising administering a therapeutically effective amount of a second therapeutic agent to the subject.
   5. The method of any one of embodiments 1 to 4, further comprising determining the level of ferritin in a sample of a subject having myelofibrosis.
   6. The method of any one of embodiments 1 to 5, wherein the momelotinib or a pharmaceutically acceptable salt thereof is administered for a treatment period of a plurality of weeks.
   7. The method of embodiment 6, wherein the treatment period of a plurality of weeks is 12 weeks or more.
   8. The method of embodiment 6, wherein the treatment period of a plurality of weeks is 24 weeks or more.
   9. The method of embodiment 6, wherein the treatment period of a plurality of weeks is 36 weeks or more.
   10. The method of any one of embodiments 1 to 9, further comprising the steps of determining the level of ferritin in a sample of the subject once a month, or twice a month, or once weekly during a treatment period.
   11. The method of embodiment 10, wherein the level of ferritin in a sample of the subject is measured once weekly.
   12. The method of embodiment 10 or 11, wherein the subject is assessed as maintaining a ferritin level within a predetermined range during the treatment period.
   13. The method of embodiment 10 or 11, wherein the subject is assessed as having a ferritin level that is not within a predetermined range during the treatment period.
   14. The method of embodiment 10 or 11, wherein the subject is assessed as having a ferritin level that is not within a predetermined range during the treatment period and wherein the method further comprises the step of terminating administration of the momelotinib or a pharmaceutically acceptable salt thereof, and/or the optional second therapeutic agent.
   15. The method of embodiment 10 or 11, wherein the subject is assessed as having a ferritin level that is not within a predetermined range during the treatment period and wherein the method further comprises the steps of
terminating administration of the momelotinib or a pharmaceutically acceptable salt thereof, and/or the optional second therapeutic agent; and
administering a second line treatment comprising:
   administering a therapeutically effective amount of a JAK inhibitor to the subject wherein the subject is assessed as having a ferritin level less than 90 ng/mL; or
   administering a therapeutically effective amount of momelotinib or a pharmaceutically acceptable salt thereof to the subject wherein the subject is assessed as having a ferritin level greater than 90 ng/mL and less than or equal to 650 ng/mL; or
   administering a therapeutically effective amount of momelotinib or a pharmaceutically acceptable salt thereof, and optionally a second therapeutic agent, to the subject wherein the subject is assessed as having a ferritin level greater than 650 ng/mL.
      16. The method of any one of embodiments 12 to 15, wherein the predetermined range of ferritin level during the treatment period is greater than or equal to 90 ng/mL, or wherein the predetermined range of ferritin level during the treatment period is greater than or equal to 90 ng/mL and less than or equal to 650 ng/mL.
      17. The method of any one of embodiments 12 to 15, wherein the predetermined range of ferritin level during the treatment period is greater than 650 ng/mL.
      18. A method of maintaining transfusion independence in a subject being treated for myelofibrosis, the method comprising
a. administering a therapeutically effective amount of a JAK inhibitor to a subject identified as (i) having myelofibrosis, (ii) being transfusion independent, and (iii) as having a ferritin level less than 90 ng/mL; or
b. administering a therapeutically effective amount of momelotinib or a pharmaceutically acceptable salt thereof to a subject identified as (i) having myelofibrosis, (ii) being transfusion independent, and (iii) as having a ferritin level greater than 90 ng/mL and less than or equal to 650 ng/mL; or
c. administering a therapeutically effective amount of momelotinib or a pharmaceutically acceptable salt thereof, and optionally a second therapeutic agent, to a subject identified as (i) having myelofibrosis, (ii) being transfusion independent, and (iii) as having a ferritin level greater than 650 ng/mL.
   19. A method of converting a subject being treated for myelofibrosis from transfusion requiring or from transfusion dependent to transfusion independent, the method comprising
a. administering a therapeutically effective amount of a JAK inhibitor to a subject identified as (i) having myelofibrosis. (ii) being transfusion requiring or transfusion dependent, and (iii) as having a ferritin level less than 90 ng/mL; or
b. administering a therapeutically effective amount of momelotinib or a pharmaceutically acceptable salt thereof to a subject identified as (i) having myelofibrosis, (ii) being transfusion requiring or transfusion dependent, and (iii) as having a ferritin level greater than 90 ng/mL;
c. administering a therapeutically effective amount of momelotinib or a pharmaceutically acceptable salt thereof to a subject identified as (i) having myelofibrosis, (ii) being transfusion requiring or transfusion dependent, and (iii) as having a ferritin level greater than 90 ng/mL and less than or equal to 650 ng/mL; or
d. administering a therapeutically effective amount of momelotinib or a pharmaceutically acceptable salt thereof, and optionally a second therapeutic agent, to a subject identified as (i) having myelofibrosis, (ii) being transfusion requiring or transfusion dependent, and (iii) as having a ferritin level greater than 650 ng/mL.
   20. The method of embodiment 18 or 19, wherein the JAK inhibitor is chosen from momelotinib or a pharmaceutically acceptable salt thereof, ruxolitinib or fedratinib.
   21. The method of embodiment 20, wherein the JAK inhibitor is momelotinib or a pharmaceutically acceptable salt thereof or ruxolitinib.
   22. The method of embodiment 20, wherein the JAK inhibitor is ruxolitinib.
   23. The method of embodiment 20, wherein the JAK inhibitor is momelotinib or a pharmaceutically acceptable salt thereof.
   24. The method of any one of embodiments 4, or 18 to 23, wherein the second therapeutic agent is a BET protein inhibitor, or is a BRD4 inhibitor.
   25. The method of embodiment 24, wherein the second therapeutic agent is chosen from GSK2820151, GSK525762, GS-5829, R06870810 (IV), BAY1238097, CC-90010, BMS-986158, 1NCB054329, 1NCB057643, ODM-207, AZD5153, FT-1101, ABBV-744, ABBV-075, PLX51107, B1894999, OTX115/MK8628, ZEN003694, RVX-000222, CPI-0610, apabetalone and fedratinib.
   26. A method of treating or preventing anemia in a subject being treated for myelofibrosis comprising
administering a therapeutically effective amount of momelotinib or a pharmaceutically acceptable salt thereof to a subject identified (i) as having myelofibrosis, and (ii) as having anemia or as being at risk for becoming anemic, and (iii) as having a ferritin level greater than or equal to 90 ng/mL.
   27. The method of any one of embodiments 18 to 26, further comprising the earlier step of determining the level of ferritin in a sample of a subject having myelofibrosis.
   28. The method of any one of embodiments 18 to 27, wherein the momelotinib or a pharmaceutically acceptable salt thereof is administered for a treatment period of a plurality of weeks.
   29. The method of embodiment 28, wherein the treatment period of a plurality of weeks is 12 weeks or more.
   30. The method of embodiment 28, wherein the treatment period of a plurality of weeks is 24 weeks or more.
   31. The method of embodiment 28, wherein the treatment period of a plurality of weeks is 36 weeks or more.
   32. The method of any one of embodiments 18 to 31, further comprising the steps of determining the level of ferritin in a sample of the subject once a month, or twice a month, or once weekly during a treatment period.
   33. The method of embodiment 32, wherein the level of ferritin in a sample of the subject is measured weekly.
   34. The method of embodiment 32 or 33, wherein the subject is assessed as maintaining a ferritin level within a predetermined range during the treatment period.
   35. The method of embodiment 32 or 33, wherein the subject is assessed as having a ferritin level that is not within a predetermined range during the treatment period.
   36. The method of embodiment 32 or 33, wherein the subject is assessed as having a ferritin level that is not within a predetermined range during the treatment period and wherein the method further comprises the step of terminating administration of the JAK inhibitor, or the momelotinib or a pharmaceutically acceptable salt thereof, and/or the optional second therapeutic agent.
   37. The method of embodiment 32 or 33, wherein the subject is assessed as having a ferritin level that is not within a predetermined range during the treatment period and wherein the method further comprises the steps of
terminating administration of the JAK inhibitor, or the momelotinib or a pharmaceutically acceptable salt thereof, and/or the optional second therapeutic agent; and
administering a second line treatment comprising:
   administering a therapeutically effective amount of a JAK inhibitor to the subject wherein the subject is assessed as having a ferritin level less than 90 ng/mL; or
   administering a therapeutically effective amount of momelotinib or a pharmaceutically acceptable salt thereof to the subject wherein the subject is assessed as having a ferritin level greater than 90 ng/mL; or
   administering a therapeutically effective amount of momelotinib or a pharmaceutically acceptable salt thereof to the subject wherein the subject is assessed as having a ferritin level greater than 90 ng/mL and less than or equal to 650 ng/mL; or
   administering a therapeutically effective amount of momelotinib or a pharmaceutically acceptable salt thereof, and optionally a second therapeutic agent, to the subject wherein the subject is assessed as having a ferritin level greater than 650 ng/mL.
      38. The method of any one of embodiments 34 to 37, wherein the predetermined range of ferritin level during the treatment period is greater than or equal to 90 ng/mL, or wherein the predetermined range of ferritin level during the treatment period is greater than or equal to 90 ng/mL and less than or equal to 650 ng/mL.
      39. The method of any one of embodiments 34 to 37, wherein the predetermined range of ferritin level during the treatment period is greater than 650 ng/mL.
      40. The method of any one of embodiments 1 to 39, wherein the subject is a human.
      41. The method of any one of embodiments 1 to 40, wherein the subject is an adult human.
      42. The method of any one of embodiments 1 to 41, wherein the subject has previously been treated with JAK inhibitor therapy other than momelotinib.
      43. The method of embodiment 42, wherein the subject has previously been treated with ruxolitinib.
      44. The method of embodiment 42 or 43, wherein the subject has had an inadequate response to or is intolerant of ruxolitinib.
      45. The method of any one of embodiments 42 to 44, wherein the subject failed to respond or ceased to respond to previous ruxolitinib therapy.
      46. The method of any one of embodiments 1 to 41, wherein the subject is naïve to JAK inhibitor therapy.
      47. The method of any one of embodiments 1 to 46, wherein the momelotinib or pharmaceutically acceptable salt thereof is momelotinib dihydrochloride salt.
      48. The method of any one of embodiments 1 to 46, wherein the momelotinib or pharmaceutically acceptable salt thereof is momelotinib dihydrochloride monohydrate.
      49. The method of any one of embodiments 1 to 46, wherein the momelotinib or pharmaceutically acceptable salt thereof is momelotinib dihydrochloride monohydrate Form 11.
      50. The method of any one of embodiments 1 to 49, wherein the momelotinib or pharmaceutically acceptable salt thereof is provided in a pharmaceutically acceptable composition.
      51. The method of any one of embodiments 1 to 50, wherein the therapeutically effective amount is between 50 mg/day and 200 mg/day.
      52. The method of embodiment 51, wherein the therapeutically effective amount is 200 mg/day, or 150 mg/day, or 100 mg/day, or 50 mg/day.
      53. The method of any one of embodiments 1 to 52, wherein the momelotinib or pharmaceutically acceptable salt thereof is administered orally.
      54. The method of any one of embodiments 1 to 53, wherein the momelotinib or pharmaceutically acceptable salt thereof is administered daily.
      55 The method of embodiment 54, wherein the momelotinib or pharmaceutically acceptable salt thereof is administered once daily.

1. A method of treating myelofibrosis in a subject, the method comprising:
administering a therapeutically effective amount of momelotinib or a pharmaceutically acceptable salt thereof to a subject identified as having (i) myelofibrosis, and (ii) a ferritin level greater than 90 ng/mL.

2. The method of claim 1, wherein the subject is identified as having a ferritin level greater than 90 ng/mL and less than 650 ng/mL.

3. The method of claim 1, wherein the subject is identified as having a ferritin level greater than 650 ng/mL.

4. The method of claim 3, further comprising administering a therapeutically effective amount of a second therapeutic agent to the subject.

5. The method of claim 1, further comprising determining the level of ferritin in a sample of a subject having myelofibrosis.

6. The method of claim 1, wherein the momelotinib or a pharmaceutically acceptable salt thereof is administered for a treatment period of a plurality of weeks.

7. The method of claim 1, further comprising steps of determining the level of ferritin in a sample of the subject once a month, or twice a month, or once weekly during a treatment period.

8. The method of claim 7, wherein the subject is assessed as maintaining a ferritin level within a predetermined range during the treatment period; or
wherein the subject is assessed as having a ferritin level that is not within a predetermined range during the treatment period; or
wherein the subject is assessed as having a ferritin level that is not within a predetermined range during the treatment period and wherein the method further comprises the step of terminating administration of the momelotinib or a pharmaceutically acceptable salt thereof, and/or the optional second therapeutic agent; or
wherein the subject is assessed as having a ferritin level that is not within a predetermined range during the treatment period and wherein the method further comprises the steps of
terminating administration of the momelotinib or a pharmaceutically acceptable salt thereof, and/or the optional second therapeutic agent; and
administering a second line treatment comprising:
administering a therapeutically effective amount of a JAK inhibitor to the subject wherein the subject is assessed as having a ferritin level less than 90 ng/mL; or
administering a therapeutically effective amount of momelotinib or a pharmaceutically acceptable salt thereof to the subject wherein the subject is assessed as having a ferritin level greater than 90 ng/mL and less than or equal to 650 ng/mL; or
administering a therapeutically effective amount of momelotinib or a pharmaceutically acceptable salt thereof, and optionally a second therapeutic agent, to the subject wherein the subject is assessed as having a ferritin level greater than 650 ng/mL.

9. The method of claim 8, wherein the predetermined range of ferritin level during the treatment period is greater than or equal to 90 ng/mL, or wherein the predetermined range of ferritin level during the treatment period is greater than or equal to 90 ng/mL and less than or equal to 650 ng/mL; or
wherein the predetermined range of ferritin level during the treatment period is greater than 650 ng/mL.

10. A method of maintaining transfusion independence in a subject being treated for myelofibrosis, the method comprising
a) administering a therapeutically effective amount of a JAK inhibitor to a subject identified as (i) having myelofibrosis, (ii) being transfusion independent, and (iii) as having a ferritin level less than 90 ng/mL; or
b) administering a therapeutically effective amount of momelotinib or a pharmaceutically acceptable salt thereof to a subject identified as (i) having myelofibrosis, (ii) being transfusion independent, and (iii) as having a ferritin level greater than 90 ng/mL and less than or equal to 650 ng/mL; or
c) administering a therapeutically effective amount of momelotinib or a pharmaceutically acceptable salt thereof, and optionally a second therapeutic agent, to a subject identified as (i) having myelofibrosis, (ii) being transfusion independent, and (iii) as having a ferritin level greater than 650 ng/mL.

11. A method of converting a subject being treated for myelofibrosis from transfusion requiring or from transfusion dependent to transfusion independent, the method comprising
a) administering a therapeutically effective amount of a JAK inhibitor to a subject identified as (i) having myelofibrosis, (ii) being transfusion requiring or transfusion dependent, and (iii) as having a ferritin level less than 90 ng/mL; or
b) administering a therapeutically effective amount of momelotinib or a pharmaceutically acceptable salt thereof to a subject identified as (i) having myelofibrosis, (ii) being transfusion requiring or transfusion dependent, and (iii) as having a ferritin level greater than 90 ng/mL;
c) administering a therapeutically effective amount of momelotinib or a pharmaceutically acceptable salt thereof to a subject identified as (i) having myelofibrosis, (ii) being transfusion requiring or transfusion dependent, and (iii) as having a ferritin level greater than 90 ng/mL and less than or equal to 650 ng/mL; or
d) administering a therapeutically effective amount of momelotinib or a pharmaceutically acceptable salt thereof, and optionally a second therapeutic agent, to a subject identified as (i) having myelofibrosis, (ii) being transfusion requiring or transfusion dependent, and (iii) as having a ferritin level greater than 650 ng/mL.

12. The method of claim 10, wherein the JAK inhibitor is chosen from momelotinib or a pharmaceutically acceptable salt thereof, ruxolitinib or fedratinib.

13. The method of claim 4, wherein the second therapeutic agent is a BET protein inhibitor, or is a BRD4 inhibitor.

14. A method of treating or preventing anemia in a subject being treated for myelofibrosis comprising
administering a therapeutically effective amount of momelotinib or a pharmaceutically acceptable salt thereof to a subject identified (i) as having myelofibrosis, and (ii) as having anemia or as being at risk for becoming anemic, and (iii) as having a ferritin level greater than or equal to 90 ng/mL.

15. The method of claim 10, further comprising in earlier step of determining the level of ferritin in a sample of a subject having myelofibrosis; and/or
wherein the momelotinib or a pharmaceutically acceptable salt thereof is administered for a treatment period of a plurality of weeks.

16. The method of claim 10, further comprising steps of determining the level of ferritin in a sample of the subject once a month, or twice a month, or once weekly during a treatment period.

17. The method of claim 16, wherein the subject is assessed as maintaining a ferritin level within a predetermined range during the treatment period; or
wherein the subject is assessed as having a ferritin level that is not within a predetermined range during the treatment period; or
wherein the subject is assessed as having a ferritin level that is not within a predetermined range during the treatment period and wherein the method further comprises the step of terminating administration of the JAK inhibitor, or the momelotinib or a pharmaceutically acceptable salt thereof, and/or the optional second therapeutic agent; or
wherein the subject is assessed as having a ferritin level that is not within a predetermined range during the treatment period and wherein the method further comprises the steps of
terminating administration of the JAK inhibitor, or the momelotinib or a pharmaceutically acceptable salt thereof, and/or the optional second therapeutic agent; and
administering a second line treatment comprising:
administering a therapeutically effective amount of a JAK inhibitor to the subject wherein the subject is assessed as having a ferritin level less than 90 ng/mL; or
administering a therapeutically effective amount of momelotinib or a pharmaceutically acceptable salt thereof to the subject wherein the subject is assessed as having a ferritin level greater than 90 ng/mL; or
administering a therapeutically effective amount of momelotinib or a pharmaceutically acceptable salt thereof to the subject wherein the subject is assessed as having a ferritin level greater than 90 ng/mL and less than or equal to 650 ng/mL; or
administering a therapeutically effective amount of momelotinib or a pharmaceutically acceptable salt thereof, and optionally a second therapeutic agent, to the subject wherein the subject is assessed as having a ferritin level greater than 650 ng/mL.

18. The method of claim 17, wherein the predetermined range of ferritin level during the treatment period is greater than or equal to 90 ng/mL, or wherein the predetermined range of ferritin level during the treatment period is greater than or equal to 90 ng/mL and less than or equal to 650 ng/mL; or
wherein the predetermined range of ferritin level during the treatment period is greater than 650 ng/mL.

19. The method of claim 1, wherein the subject has previously been treated with JAK inhibitor therapy other than momelotinib; or
wherein the subject has previously been treated with ruxolitinib.

20. The method of claim 19, wherein the subject has had an inadequate response to or is intolerant of ruxolitinib; and/or
wherein the subject failed to respond or ceased to respond to previous ruxolitinib therapy; and/or
wherein the subject is naïve to JAK inhibitor therapy.

21. The method of claim 1, wherein the therapeutically effective amount is between 50 mg/day and 200 mg/day.
